(19) United States
(12) Patent Application Publication
(10) Pub. No.: US 2005/0260701 A1
(43) Pub. Date: Nov. 24, 2005

(54) Method for enzymatic production of GLP-1 (7-36) amide peptides

(76) Inventors: Fred W. Wagner, Walton, NE (US); Peng Luan, Fishers, IN (US); Yuannan Xia, Lincoln, NE (US); Mary J. Bossard, Madison, AL (US); Barton Holmquist, Eagle, NE (US); Edwin H. Merrifield, Lynnwood, WA (US); Daniel Strydom, Lincoln, NE (US)

Correspondence Address: SCHWEGMAN, LUNDBERG, WOESSNER & KLUTH, P.A., P.O. BOX 2938, MINNEAPOLIS, MN, 55402-0938, US

(21) Appl. No.: 10/997,061

(22) Filed: Nov. 24, 2004

Related U.S. Application Data
(63) Continuation of application No. PCT/US03/16469, filed on May 23, 2003.
(60) Provisional application No. 60/383,214, filed on May 24, 2002.

(51) Int. Cl. C12P 21/06; C07K 14/605

(52) U.S. Cl. 435/68.1; 530/399

(57) ABSTRACT

The invention provides methods for making peptides from a polypeptide containing at least one copy of the peptide using clostripain to excise the peptide from the polypeptide. The methods enable the use of a single, highly efficient enzymatic cleavage to produce any desired peptide sequence.

### BACKGROUND OF THE INVENTION

Although bioactive peptides can be produced chemically by a variety of synthesis strategies, recombinant technology offers the potential for inexpensive, large-scale production of peptides without the use of organic solvents, highly reactive reagents or potentially toxic chemicals. However, expression of short peptides in Escherichia coli and other microbial systems can sometimes be problematic. For example, short peptides are often degraded by the proteolytic and metabolic enzymes present in microbial host cells. Use of a fusion protein to carry the peptide of interest may help avoid cellular degradation processes because the fusion protein is large enough to protect the peptide from proteolytic cleavage. Moreover, certain fusion proteins can direct the peptide to specific cellular compartments, i.e. cytoplasm, periplasm, inclusion bodies or media; thereby helping to avoid cellular degradation processes. However, while use of a fusion protein may solve certain problems, cleavage and purification of the peptide away from the fusion protein can give rise to a whole new set of problems.

Preparation of a peptide from a fusion protein in pure form requires that the peptide be released and recovered from the fusion protein by some mechanism. In many cases, the peptide of interest forms only a small portion of the fusion protein. For example, many peptidyl moieties are fused with β-galactosidase that has a molecular weight of about 100,00 daltons. A peptide with a molecular weight of about 3000 daltons would only form about 3% of the total mass of the fusion protein. Also, separate isolation or purification procedures (e.g. affinity purification procedures) are generally required for each type of peptide released from a fusion protein. Release of the peptide from the fusion protein generally involves use of specific chemical or enzymatic cleavage sites that link the carrier protein to the desired peptide (Forsberg et al., I. J. Protein Chem., 11:201-211, (1992)). Chemical or enzymatic cleavage agents employed for such cleavages generally recognize a specific sequence. However, if that cleavage sequence is present in the peptide of interest, then a different cleavage agent must usually be employed. Use of a complex fusion partner (e.g. β-galactosidase) that may have many cleavage sites produces a complex mixture of products and complicates isolation and purification of the peptide of interest.

Chemical cleavage reagents in general recognize single or paired amino acid residues that may occur at multiple sites along the primary sequence, and therefore may be of limited utility for release of large peptides or protein domains which contain multiple internal recognition sites. However, recognition sites for chemical cleavage can be usefull at the junction of short peptides and carrier proteins. Chemical cleavage reagents include cyanogen bromide, which cleaves at methionine residues (Piers et al., Gene, 134:7 (1993)), N-chloro succinimide (Forsberg et al., Biofactors, 2:105 (1989)) or BNPS-skatole (Knott et al., Eur. J. Biochem., 174:405 (1988); Dykes et al., Eur. J Biochem., 174:411 (1988)) which cleave at tryptophan residues, dilute acid which cleaves aspartyl-prolyl bonds (Grain et al., Bio/Technology 12:1017 (1994); Marcus, Int. J. Peptide Protein Res., 25:542 (1985)), and hydroxylamine which cleaves asparagine-glycine bonds at pH 9.0 (Moks et al., Bio/Technology. 5:379 (1987)).

For example, Shen describes bacterial expression of a fusion protein encoding pro-insulin and β-galactosidase within insoluble inclusion bodies where the inclusion bodies were first isolated and then solubilized with formic acid prior to cleavage with cyanogen bromide. (Shen, Proc. Nat'l. Acad. Sci. (USA), 281:4627 (1984)). Dykes et al. describes soluble intracellular expression of a fusion protein encoding α-human atrial natriuretic peptide and chloramphenicol acetyltransferase in E. coli where the fusion protein was chemically cleaved with 2-(2-nitrophenylsulphenyl)-methyl-3′-bromoindolenine to release peptide. (Dykes et al., Eur. J. Biochem., 174: 11 (1988)). Ray et al. describes soluble intracellular expression in E. coli of a fusion protein encoding salmon calcitonin and glutathione-S-transferase where the fusion protein was cleaved with cyanogen bromide (Ray et al., Bio/Technology, 11:64 (1993)).

Proteases can provide gentler cleavage conditions and sometimes even greater cleavage specificity than chemical cleavage reagents because a protease will often cleave a specific site defined by the flanking amino acids and the protease can often perform the cleavage under physiological conditions. For example, Schellenberger et al. describes expression of a fusion protein encoding a substance P peptide (11 amino acids) and β-galactosidase within insoluble inclusion bodies, where the inclusion bodies were first isolated and then treated with chymotrypsin to cleave the fusion protein. (Schellenberger et al., Int. J. Peptide Protein Res., 41:326 (1993)). Pilon et al. describe soluble intracellular expression in E. coli of a fusion protein encoding a peptide and ubiquitin where the fusion protein was cleaved-with a ubiquitin specific protease, UCH-L3. (Pilon et al., Biotechnol. Prog., 13:374 (1997)). U.S. Pat. No. 5,595,887 to Coolidge et al. discloses generalized methods of cloning and isolating peptides. U.S. Pat. No. 5,707,826 to Wagner et al. describes an enzymatic method for modification of recombinant polypeptides.

Glucagon Like Peptides, GLP-1 and GLP-2, are encoded by the proglutagon gene. In vivo, the glucagon gene expresses a 180 amino acid prepropolypeptide that is proteolytically processed to form glucagon, two forms of GLP-1 and GLP-2. The original sequencing studies indicated that GLP-1 possessed 37 amino acid residues. However, subsequent information showed that this peptide was a propeptide and was additionally processed to remove 6 amino acids from the amino-terminus to a form GLP-1(7-37), an active form of GLP-1. The glycine at position 37 is also transformed to an amide in vivo to form GLP-1(7-36)amide. GLP-1(7-37) and GLP-1(7-36)amide are insulinotropic hormones of equal potency.

The recombinant production of any of these GLP peptides in high yield, however, is elusive because post expression manipulation using traditional methods provides poor results. Consequently, the goal of recombinant production of GLP peptides through a one pot, high yield process lends itself to protease post-expression manipulation. Cleavage of possible pre-GLP polypeptide substrates by currently available processes necessitate use of different proteases and unique conditions and/or pre-or post-manipulation of the precursor polypeptides. Hence, improved and simplified methods for making GLP peptides are needed. In particular, a simplified, high yield method for making GLP peptides is needed.

### SUMMARY OF THE INVENTION

These and other needs are achieved by the present invention, which is directed to a site specific clostripain cleavage of a single or multicopy polypeptide having or containing a peptide sequence of the Formula GLP-1 (7-36), GLP-1 (7-36) amide, or GLP-1 (7-37) as well as conservative substitutions thereof (hereinafter these peptides are termed the GLP-1 peptides as a group). In particular, the present invention is directed to a method that surprisingly selects a particular clostripain cleavage site from among several that are present in a single or multicopy polypeptide incorporating the amino acid sequence of GLP-1 peptides. The result of this surprising characteristic of the method of the invention is the development of a versatile procedure for the production of the desired GLP-1 peptides from a single or multicopy polypeptide.

An especially preferred method according to the invention involves the production of the desired peptide through recombinant techniques. This feature is accomplished through use of a single copy polypeptide having a discardable sequence ending in arginine joined to the N-terminus of the desired peptide. The cleavage of that designated arginine according to the invention is so selective that the desired peptide may contain any sequence of amino acids. The cleavage produces a single copy of the desired peptide. Thus, the methods according to the invention enable the production of the desired GLP-1 peptides. Some of the salient details of these methods of the invention are summarized in the following passages.

The invention provides methods for making peptides using clostripain cleavage of a larger polypeptide that has at least one copy of the desired GLP-1 (7-36) peptide. According to the invention, clostripain recognizes a polypeptide having a site as indicated in Formula I and cleaves a peptide bond between amino acids Xaa₂ and Xaa₃:
Xaa₁-Xaa₂-Xaa₃ Formula I
wherein Xaa₁ and Xaa₃ in general may be any non-acidic amino acid residue and Xaa₂ is arginine. According to a preferable aspect of the invention, clostripain selectively recognizes the site as indicated in Formula I and cleaves the peptide bond between amino acids Xaa₂ and Xaa₃; therein Xaa₁ is an amino acid residue with an acidic side chain such as aspartic acid, or glutamic acid, or a non-acidic side chain such as proline or glycine; Xaa₂ is arginine; and Xaa₃is not an acidic amino acid. Also, through the control of any one or more of pH, time, temperature and reaction solvent involved in the cleavage reaction, the rate and selectivity of the clostripain cleavage may be manipulated. Thus, for example, the GLP-1 (7-36) peptide of the-sequence.

HAEGTFTSDVSSYLEGQAAKEFIAWLVKGR, (SEQ ID: 1), may be formed as multiple copies coupled together with a linker of an appropriate sequence, or multiple copies coupled together in tandem with the N-terminal histidine (His) forming a peptide bond with the C-terminal arginine (Arg) of the upstream copy, or a-discardable sequence ending with Xaa₁-Xaa₂-Xaa₃ coupled to the N-terminus, or beginning with Xaa₃ coupled to the C-terminus, of the desired peptide.

Clostripain will eventually cleave the peptide bond on the carboxyl side of any arginine or lysine appearing in an amino acid sequence irrespective of the amino acid residues adjacent to arginine. Surprisingly, it has been discovered that the rate of clostripain cleavage of a polypeptide can be dramatically altered by specifically altering amino acids immediately on the N-terminal and C-terminal side of an arginine residue that acts as a clostripain cleavage site. In particular, according to the invention, this preferred clostripain cleavage of an argmine—amino acid residue peptide bond can be manipulated to be highly selective through use of an acidic amino acid residue bonded to the amine side of argine, eg. Xaa₁ of foregoing Formula I. According to the invention, it has also been discovered that by manipulation of any one or more of pH, time, temperature and solvent character, the rate of clostripain cleavage can be manipulated to affect cleavage of a selected Xaa₂-Xaa₃ peptide bond of Formula I. Combinations of these factors will enable selection of particular arginine—amino acid residue bonds from among several differing such bonds that may be present in the precursor polypeptide.

In one aspect, the invention provides a method for producing the desired peptide from a polypeptide by cleaving at least one peptide bond within the polypeptide using clostripain. The clostripain cleaves a peptide bond between amino acids Xaa₂ and Xaa₃ of a polypeptide having the Formula II:
(Xaa₃-Peptide₁-Xaa₁-Xaa₂)ₙ-Xaa₃-Peptide₁-Xaa₁-Xaa₂ Formula II
In this aspect of the invention, the desired GLP-1 (7-36) peptide has the Formula Xaa₃-Peptide₁-Xaa₁-Xaa₂ wherein Xaa₃ is His, Xaa₁ is Gly and Xaa₂ is Arg. Also in this aspect of the invention, n is an integer ranging from 0 to 50.

In another aspect, the invention provides an alternative method for producing the desired peptide GLP-1 (7-36). Such a method involves cleaving with clostripain a peptide bond between amino acids Xaa₂ and Xaa₃ within a polypeptide comprising Formula III:
(Linker-Xaa₃-Peptide₁)ₙ-Linker-Xaa₃-Peptide₁ Formula III
In this aspect of the invention, the desired peptide GLP-1 (7-36) has the Formula Xaa₃-Peptide₁, and n is an integer ranging from 0 to 50. Xaa₃ is H. Linker refers to a cleavable peptide linker having Formula IV:
(Peptide₅)ₘ-Xaa₁-Xaa₂ Formula IV
m is an integer ranging from 0 to 50. Xaa₁ is aspartic acid, glycine, proline or glutamic acid. Xaa₂ is arginine. Peptide₅ is any single or multi-amino acid sequence not containing the sequence Xaa₁-Xaa₂, such as histidine. For example Formula III may read
His-Gly-Arg-GLP-1(7-34)-Gly-Arg-His-Gly-Arg-GLP-1(7-34)-Gly-Arg (SEQ ID NO:28).

The invention further provides a method of producing a GLP-1 (7-36) peptide. The method involves the steps of
(a) recombinantly producing a polypeptide of the Formula VI:
   Tag-Linker-[GLP-1 (7-36)]_{q} Formula VI
   wherein Tag is an amino acid sequence having SEQ ID NO:17 or 18; Linker is a cleavable peptide linker of Formula IV described above; GLP-1(7-36) has SEQ ID NO:1; and q is an integer of about 2 to about 20;
(b) isolating the polypeptide of Formula VI; and
(c) cleaving at least one peptide bond within the polypeptide of Formula VI using clostripain, wherein clostripain cleaves a peptide bond on the C-terminal side of Xaa₂.
   The invention also includes methods of transpeptidation and C-terminus amidation. In particular, this method of the invention provides a method of producing a GLP-1(7-36)NH₂ peptide having SEQ ID NO:2. The steps include
(a) recombinantly producing a polypeptide of the Formula VII:
   Tag-Linker-[GLP-1(7-36)-Linker₂]_{q} VIII
   wherein:
Tag is an amino acid sequence comprising SEQ ED NO:17 or 18;
Linker is a cleavable peptide linker having Formula IV:
   (Peptide₅)ₘ-Xaa₁-Xaa₂ IV
   wherein:
n is an integer ranging from 0 to 50;
m is an integer ranging from 0 to 50;
Xaa₁ is aspartic acid, glycine, proline or glutamic acid;
Xaa₂ is arginine; and
Peptide₅ is a single or pair of amino acid residues;
Linker₂ is SEQ ID NO:23;
GLP-1(7-36) has SEQ ID NO:1;
q is an integer of about 2 to about 20;
(b) isolating the polypeptide of Formula VIII;
(c) cleaving at least one peptide bond within the polypeptide of Formula VII using clostripain in the presence of ammonia, wherein clostripain cleaves a peptide bond on the C-terminal side of Xaa₂, amidates the carbonyl of Xaa₂ and thereby forms a GLP-1(7-36)NH₂ peptide having SEQ ID NO:2. Alternatively, glycine instead of ammonia can be included within the clostripain cleavage to produce a GLP-1 (7-37) peptide.

Another example according to the invention provides a method of producing a GLP-1(7-36)(K26R)—NH₂ peptide having SEQ ID NO:6. The steps include
(a) recombinantly producing a polypeptide of the Formula VIII:
   Tag-Linker-[GLP-1(7-36)(K26R)-Linker₂]_{q} VIII
   wherein:
Tag is an amino acid sequence comprising SEQ ID NO:17 or 18;
Linker is a cleavable peptide linker having Formula IV:
   (Peptide₅)ₘ-Xaa₁-Xaa₂ IV
   wherein:
n is an integer ranging from 0 to 50;
m is an integer ranging from 0 to 50;
Xaa₁ is aspartic acid, glycine, proline or glutamic acid;
Xaa₂ is arginine; and
Xaa₄ and Xaa₅ are separately any amino acid;

GLP-1(7-36)(K26R) has SEQ ID NO:5;
q is an integer of about 2 to about 20;
(b) isolating the polypeptide of Formula VIII;
(c) cleaving at least one peptide bond within the polypeptide of Formula VIII using clostripain, wherein clostripain cleaves a peptide bond on the C-terminal side of Xaa₂, amidates the carbonyl of Xaa₂ and thereby forms a GLP-1(7-36)(K26R)NH₂ peptide having SEQ ID NO:6.

Finally, additional aspects of the invention include modifications regarding production of a polypeptide within a bacterial cell. A DNA segment encoding the precursor polypeptide can be transformed into the bacterial host cells. The DNA segment can also encode a peptidyl sequence linked to the precursor polypeptide wherein the peptidyl sequence encourages the polypeptide to be sequestered within bacterial inclusion bodies. Such peptidyl sequences are termed “inclusion body leader partners” and include peptidyl sequences having, for example, SEQ ID NO:19, 20, 21 or 22. Use of such an inclusion body leader partner facilitates isolation and purification of the polypeptide. Isolation of the bacterial inclusion bodies containing the polypeptide is simple (e.g. centrifugation). According to the invention, the isolated inclusion bodies can be used without substantial purification, for example, by solubilizing the polypeptide in urea and then conducting the clostripain cleavage reaction either before or after removal of the urea. Clostrpain is capable of cleaving a polypeptide in comparatively high concentrations of urea, for examples in the presence of about 0 M to about 8 M urea, so removal of urea is not required. Hence, the invention provides methods for cleaving a soluble polypeptide, or an insoluble polypeptide that can be made soluble by adding urea.

### DESCRIPTION OF THE DRAWINGS

FIG. 1 provides a schematic diagram of a pBN121 based vector containing a DNA segment encoding the precursor polypeptide T7-tag-GS-[GPGDR-GLP-1(7-36)-AFL]₃pYX (SEQ ID NO:9), Chlorella promotor.

FIG. 2 illustrates a typical growth curve of recombinant E. coli. Addition of IPTG generally occurs between 10 and 11 hours. Cells are harvested 6-10 hours after IPTG induction.

FIGS. 3A-3C illustrate HPLC analysis of the T7-tag-GS-[GPGDR-GLP-1(7-36)-AFL]₃ (SEQ ID NO:9) precursor polypeptide (about 16-20 gm/L) from a typical fermentation. Cell samples were taken after 10 hours of induction and prepared for analysis as described in the text.

FIGS. 4A-4H show the LC/MS identification of T7-tag-GS-[GPGDR-GLP-1(7-36)-AFL]₃ (SEQ ID NO:9) clostripain digestion products; A) Total ion chromatogram. Peaks 1-4 represent the major cleavage products after clostripain digestion. The major mass signals represent +2 and +3 charges; B) TV chromatogram at 280 nm; C) GLP-1(-18 amu); D) GLP-1: amide of GLP-1 (7-36); E) GLP-1 (OH): GLP-1(7-36) free acid; F) contains both GLP-1-AFLGPDR:GLP-1 (SEQ ID NO:29) with a linker still attached and GLP-1(7-34); H) shows an HPLC analysis of purified GLP-1(7-36)NH₂.

FIG. 5 illustrates the production of the C-terminal amidated cleavage product GLP-1(7-36)-NH₂. Peak (1) is T7-tag-GS-[GPGDR-GLP-1(7-36)-AFL]₃ (SEQ ID NO:9) at time 0. Peak (2) is GLP-1(7-36)-NH₂ after clostripain digestion, Peak (3) is GLP-1(7-36)-OH after clostripain digestion.

FIGS. 6A-6C show the production of GLP-1(7-37) as identified by LC/MS analysis. (A) GLP-1(7-36) AFAHSe (Homoserine and lactone mixture) (SEQ ID NO:10) (B) is GLP-1(7-37) and (C) is the LC-MS showing the correct mass (3356 AMU) for GLP-1(7-37).

### DEFINITIONS OF THE INVENTION

Abbreviations: LC-MS: liquid chromatography-mass spectroscopy; TFA: trifloroacetic acid; DTT: dithiotlreitol; DTE: dithioerythritol.

An “amino acid analog” includes amino acids that are in the D rather than L form, genetically encoded, non-genetically encoded, synthetic amino acids and amino acid analogs.

An “Amino acid analog” includes amino acids that are in the D rather than L form, as well as other well known amino acid analogs, e.g., N-alkyl amino acids, lactic acid, and the like. These analogs include phosphoserine, phosphothreonine, phosphotyrosine, hydroxyprolinie, gamma-carboxyglutamate; hippuric acid, octahydroindole-2-carboxylic acid, statine, 1,2,3,4,-tetrahydroisoquinoline-3-carboxylic acid, penicillamine, ornithine, citruline, N-methyl-alanine, para-benzoyl-phenylalanine, phenylglycine, propargylglycine, sarcosine, N-acetylserine, N-formylmethionine, 3-methylhistidine, 5-hydroxylysine, norleucine, norvaline, orthonitrophenylglycine and other similar amino acids.

The terms, “cells,” “cell cultures”, “recombinant host cells”, “host cells”, and other such terms denote, for example, microorganisms, insect cells, and mammalian cells, that can be, or have been, used as recipients for nucleic acid constructs or expression cassettes, and include the progeny of the original cell which has been transformed. It is understood that the progeny of a single parental cell may not necessarily be completely identical in morphology or in genomic or total DNA complement as the original parent, due to natural, accidental, or deliberate mutation. Many cells are available from ATCC and commercial sources. Many mammalian cell lines are known in the art and include, but are not limited to, Chinese hamster ovary (CHO) cells, HeLa cells, baby hamster kidney (BHK) cells, monkey kidney cells (COS), and human hepatocellular carcinoma cells (e.g., Hep G2). Many prokaryotic cells are known in the art and include, but are not limited to, Escherichia coli and Salmonella typhimurium. Sambrook and Russell, Molecular Cloning: A Laboratory Manual, 3rd edition (Jan. 15, 2001) Cold Spring Harbor Laboratory Press, ISBN: 0879695765. Many insect cells are known in the art and include, but are not limited to, silkworm cells and mosquito cells. (Franke and Hruby, J. Gen. Virol., 66:2761 (1985); Marumoto et al., J. Gen. Virol., 68:2599 (1987)).

A “cleavable peptide linker” refers to a peptide sequence having a clostripain cleavage recognition sequence.

A “coding sequence” is a nucleic acid sequence that is translated into a polypeptide, such as a preselected polypeptide, usually via mRNA. The boundaries of the coding sequence are determined by a translation start codon at the 5′-terminus and a translation stop codon at the 3′-terminus of an mRNA. A coding sequence can include, but is not limited to, cDNA, and recombinant nucleic acid sequences.

A “conservative amino acid” refers to an amino acid that is functionally similar to a second amino acid. Such amino acids maybe substituted for each other in a polypeptide with minimal disturbance to the structure or function of the polypeptide. The following five groups each contain amino acids that are conservative substitutions for one another: Aliphatic: Glycine (G), Alanine (A), Valine (V), Leucine (L), Isoleucine (I); Aromatic: Phenylalanine (F), Tyrosine (Y), Tryptophan (W); Sulfur-containing: Methionine (M), Cysteine (C); Basic: Arginine (R), Lysine (K), Histidine (H); Acidic: Aspartic acid (D), Glutamic acid (E), Neutral: Asparagine (N), Glutamine (Q). Examples of other synthetic and non-genetically encoded amino acid types are provided herein.

The term “gene” is used broadly to refer to any-segment of nucleic acid that encodes a preselected polypeptide. Thus, a gene may include a coding sequence for a preselected polypeptide and/or the regulatory sequences required for expression. A gene can be obtained from a variety of sources. For example, a gene can be cloned or PCR amplified from a source of interest, or it can be synthesized from known or predicted sequence information.

An “inclusion body” is an amorphous polypeptide deposit in the cytoplasm of a cell. In general, inclusion bodies comprise aggregated protein that is improperly folded or inappropriately processed.

An “inclusion body leader partner” is a peptide that causes a polypeptide to which it is attached to form an inclusion body when expressed within a bacterial cell. The inclusion body leader partners of the invention can be altered to confer isolation enhancement onto an inclusion body that contains the altered inclusion body leader partner.

The term “lysate” as used herein refers to the product resulting from the breakage of cells. Such cells include both prokaryotic and eukaryotic cells. For example, bacteria may be lysed though a large number of art recognized methods. Such methods include, but are not limited to, treatment of cells with lysozyme, French press, treatment with urea, organic acids, and freeze thaw methods. Methods for lysing cells are known and have been described. (Sambrook and Russell, Molecular Cloning: A Laboratory Manual, 3rd edition (Jan. 15, 2001) Cold Spring Harbor Laboratory Press, ISBN: 0879695765; Stratagene, La Jolla, Calif.).

An “open reading frame” (ORF) is a region of a nucleic acid sequence that encodes a translatable polypeptide.

“Operably-linked” refers to the association of nucleic acid sequences or amino acid sequences on a single nucleic acid fragment or a single amino acid sequence so that the function of one is affected by the other. For example, a regulatory DNA sequence is said to be “operably linked to” or “associated with” a DNA sequence that codes for an RNA if the two sequences are situated such that the regulatory DNA sequence affects expression of the coding DNA sequence (i.e., that the coding sequence or functional RNA is under the transcriptional control of the promoter). In an example related to amino acid sequences, an inclusion body leader partner is said to be operably linked to a preselected amino acid sequence why the inclusion body leader partner causes a leader protein to form an inclusion body. In another example, a signal sequence is said to be operably linked to a preselected amino acid when the signal sequence directs the leader protein to a specific location in a cell.

The term “polypeptide” refers to a polymer of amino acids and does not limit the size to a specific length of the product. However, as used herein, a polypeptide is generally longer than a peptide and may include one or more copies of a peptide of interest (the terms polypeptide of interest and desired peptide are used synonymously herein). This term also optionally includes post expression modifications of the polypeptide, for example, glycosylations; acetylations, phosphorylations and the lice. Included within the definition are, for example, polypeptides containing one or more analogues of an amino acid or labeled amino acids

“Promoter” refers to a nucleotide sequence, usually upstream (5′) to its coding sequence, which controls the expression of the coding sequence by providing the recognition for RNA polymerase and other factors required for proper transcription. “Promoter” includes a minimal promoter that is a short DNA sequence comprised of a TATA-box and other sequences that serve to specify the site of transcription initiation, to which regulatory elements are added for control of expression. “Promoter” also refers to a nucleotide sequence that includes a minimal promoter plus regulatory elements that is capable of controlling the expression of a coding sequence. Promoters may be derived in their entirety from a native gene, or be composed of different elements derived from different promoters found in nature, or even be comprised of synthetic DNA segments. A promoter may also contain DNA segments that are involved in the binding of protein factors that control the effectiveness of transcription initiation in response to physiological or environmental conditions.

The term “purification stability” refers to the isolation characteristics of an inclusion body formed from a polypeptide having an inclusion body leader partner operably linked to a polypeptide. High purification stability indicates that an inclusion body can be isolated from a cell in which it was produced. Low purification stability indicates that the inclusion body is unstable during purification due to dissociation of the polypeptides forming the inclusion body.

When referring to a polypeptide or nucleic acid, “isolated” means that the polypeptide or nucleic acid has been removed from its natural source. An isolated polypeptide or nucleic acid may be present within a non-native host cell and so the polypeptide or nucleic acid is therefore not necessarily “purified.”

The term “purified” as used herein preferably means at least 75% by weight, more preferably at least 85% by weight, more preferably still at least 95% by weight, and most preferably at least 98% by weight, of biological macromolecules of the same type present (but water, buffers, and other small molecules, especially molecules having a molecular weight of less than 1000, can be present).

“Regulated promoter” refers to a promoter that directs gene expression in a controlled manner rather than in a constitutive manner. Regulated promoters include inducible promoters and repressible promoters. Such promoters may include natural and synthetic sequences as well as sequences that may be a combination of synthetic and natural sequences. Different promoters may direct the expression of a gene in response to different environmental conditions. Typical regulated promoters useful in the invention include, but are not limited to, promoters used to regulate metabolism (e.g. an IPTG-inducible lac promoter) heat-shock promoters (e.g. an SOS promoter), and bacteriophage promoters (e.g. a T7 promoter).

A “ribosome-binding site” is a DNA sequence that encodes a site on an mRNA at which the small and large subunits of a ribosome associate to form an intact ribosome and initiate translation of the mRNA. Ribosome binding site consensus sequences include AGGA or GAGG and are usually located some 8 to 13 nucleotides upstream (5′) of the initiator AUG codon on the mRNA. Many ribosome-binding sites are known in the art. (Shine et al., Nature, 254: 34, (1975); Steitz et al., “Genetic signals and nucleotide sequences in messenger RNA”, in: Biological Regulation and Development: Gene Expression (ed. R. F. Goldberger) (1979)).

A “selectable marker” is generally encoded on the nucleic acid being introduced into the recipient cell. However, co-transfection of a selectable marker can also be used during introduction of nucleic acid into a host cell. Selectable markers that can be expressed in the recipient host cell may include, but are not limited to, genes which render the recipient host cell resistant to drugs such as actinomycin C₁, actinomycin D, amphotericin, ampicillin, bleomycin, carbenicillin, chloramphenicol, geneticin, gentaiuycin, hygromycin B, kanamycin monosulfate, methotrexate, mitomycin C, neomycin B sulfate, novobiocin sodium salt, penicillin G sodium salt, puromycin dihydrochloride, rifampicin, streptomycin sulfate, tetracycline hydrochloride, and erythromycin. (Davies et al., Ann. Rev. Microbiol., 32: 469, 1978). Selectable markers may also include biosynthetic genes, such as those in the histidine, tryptophan, and leucine biosynthetic pathways. Upon transfection or transformation of a host cell, the cell is placed into contact with an appropriate selection marker.

The term “self-adhesion” refers to the association between polypeptides that have an inclusion body leader partner to form an inclusion body. Self-adhesion may affect the purification stability of an inclusion body formed from the polypeptide. Self-adhesion that is too great produces inclusion bodies having polypeptides that are so tightly associated with each other that it is difficult to separate individual polypeptides from an isolated inclusion body. Self-adhesion that is too low produces inclusion bodies that are unstable during isolation due to dissociation of the polypeptides that form the inclusion body. Self-adhesion can be regulated by altering the amino acid sequence of an inclusion body leader partner.

A “Signal sequence” is a region in a protein or polypeptide responsible for directing an operably linked polypeptide to a cellular location or compartment designated by the signal sequence. For example, signal sequences direct operably linked polypeptides to the inner membrane, periplasmic space, and outer membrane in bacteria. The nucleic acid and amino acid sequences of such signal sequences are well known in the art and have been reported. (Watson, Molecular Biology of the Gene, 4th edition, Benjamin/Cummings Publishing Company, Inc., Menlo Park, Calif. (1987); Masui et al., in: Experimental Manipulation of Gene Expression, (1983); Ghrayeb et al., EMBO J., 3: 2437 (1984); Oka et al., Proc. Natl. Acad. Sci. USA, 82: 7212 (1985); Palvaet al., Proc. Natl. Acad. Sci. USA, 79: 5582 (1982); U.S. Pat. No. 4,336,336).

Signal sequences, preferably for use in insect cells, can be derived from genes for secreted insect or baculovirus proteins, such as the baculovirus polyhedrin gene (Carbonell et al., Gene 73: 409 (1988)). Alternatively, since the signals for mammalian cell posttranslational modifications (such as signal peptide cleavage, proteolytic cleavage, and phosphorylation) appear to be recognized by insect cells, and the signals required for secretion and nuclear accumulation also appear to be conserved between the invertebrate cells and vertebrate cells, signal sequences of non-insect origin, such as those derived from genes encoding human a-interferon (Maeda et al., Nature, 315:592 (1985)), human gastrin-releasing-peptide (Lebacq-Verheyden et al., Mol. Cell. Biol., 8: 3129 (1988)), human IL-2 (Smith et al., Proc. Natl. Acad. Sci. USA, 82: 8404 (1985)), mouse IL-3 (Miyajima et al., Gene. 58: 273 (1987)) and human glucocerebrosidase (Martin et al., DNA, 7: 99 (1988)), can also be used to provide for secretion in insects.

The term “solubility” refers to the amount of a substance that can be dissolved in a unit volume of solvent. For example, solubility as used herein refers to the ability of a polypeptide to be dissolved in a volume of solvent, such as a biological buffer.

A “Tag” sequence refers to an amino acid sequence that is operably linked to the N-terminus of a peptide or protein. Such tag sequences may provide for the increased expression of a desired peptide or protein. Such tag sequences may also form a cleavable peptide linker when they are operably linked to another peptide or protein. Examples of tag sequences include, but are not limited to, the sequences indicated in SEQ ID NOs: 17 and 18.

A “transcription terminator sequence” is a signal within DNA that functions to stop RNA synthesis at a specific point along the DNA template. A transcription terminator may be either rho factor dependent or independent. An example of a transcription terminator sequence is the T7 terminator. Transcription terminators are known in the art and may be isolated from commercially available vectors according to recombinant methods known in the art. (Sambrook and Russell, Molecular Cloning: A Laboratory Manual 3rd edition (Jan. 15, 2001) Cold Spring Harbor Laboratory Press, ISBN: 0879695765; Stratagene, La Jolla, Calif.).

“Transformation” refers to the insertion of an exogenous nucleic acid sequence into a host cell, irrespective of the method used for the insertion. For example, direct uptake, transduction, f-mating or electroporation may be used to introduce a nucleic acid sequence into a host cell. The exogenous nucleic acid sequence may be maintained as a non-integrated vector, for example, a plasmid, or alternatively, may be integrated into the host genome.

A “translation initiation sequence” refers to a DNA sequence that codes for a sequence in a transcribed mRNA that is optimized for high levels of translation initiation. Numerous translation initiation sequences are known in the art. These sequences are sometimes referred to as leader sequences. A translation initiation sequence may include an optimized ribosome-binding site. In the present invention, bacterial translational start sequences are preferred. Such translation initiation sequences are available in the art and may be obtained from bacteriophage T7, bacteriophage. 10, and the gene encoding ompT. Those of skill in the art can readily obtain and clone translation initiation sequences from a variety of commercially available plasmids, such as the pET (plasmid for expression of T7 RNA-polymerase) series of plasmids. (Stratagene, La Jolla, Calif.).

A “unit” of clostripain activity is defined as the amount of enzyme required to transform 1 μmole of benzoyl-L-arginine ethyl ester (BAEE) to benzoyl-L-arginine per minute at 25° C. under defined reaction conditions. The transformation is measured spectroscopically at 253 nm. The assay solution was comprised of 0.25 mM BAEE, 10 mM HEPES (pH 7.6), 2 mM CaCl₂, and 2.5 mM DTT.

A “variant” polypeptide is a polypeptide derived from a reference polypeptide by deletion, substitution or addition of one or more amino acids to the N-terminal and/or C-terminal end of the reference polypeptide; deletion or addition of one or more amino acids at one or more sites in the reference protein; or substitution of one or more amino acids at one or more sites in the reference protein. Such substitutions or insertions are preferably conservative amino acid substitutions. Methods for such manipulations are generally known in the art. Kunkel, Proc. Natl. Acad. Sci. USA, 82:488, (1985); Kunkel et al., Methods in Enzymol., 154:367 (1987); U.S. Pat. No. 4,873,192; Walker and Gaastra, eds. (1983) Techniques in Molecular Biology (MacMillan Publishing Company, New York) and the references cited therein. Guidance as to appropriate amino acid substitutions that do not affect biological activity of the protein of interest may be found in the model of Dayhoffet al. (1978) Atlas of Protein Sequence and Structure (Natl. Biomed. Res. Found., Washington, D.C.).

### DETAILED DESCRIPTION OF THE INVENTION

The invention provides methods for efficiently making peptides of the Formulas GLP-1 (7-36), GLP-1 (7-36 amide), GLP-1 (7-37) as well as conservative substitutions thereof. The peptides are made using recombinant and proteolytic procedures. The invention enables the wide-ranging use of a single cleavage enzyme whose selectivity can be manipulated. In particular, the enzyme, clostripain, can be manipulated to cleave a particular site when the same primary cleavage site appears elsewhere in the peptide. Although limited to initial cleavage at a C-terminal side of arginine residues, the method provides versatility. The versatility arises from the surprising ability to manipulate clostripain so that it will cleave at the C-terminus even though arginine or lysine appears elsewhere within the peptide sequence.

The need to avoid reassimilation of an expressed, desired peptide by host expression cells dictates that the desired peptide should have a significantly high molecular weight and varied amino acid sequence. Such peptide features are desirable when recombinant peptides are being produced. This need means that the expressed polypeptide be formed either as a multicopy of the desired peptide or as a combination of the desired peptide linked to a discardable peptide sequence. Use of the former multicopy scheme provides multiple copies of the desired peptide under certain circumstances and the desired peptide with several additional amino acid residues at its N and C termini under all other circumstances. Use of the latter single copy scheme provides at least a single copy of the desired peptide.

According to the invention, the latter scheme may be employed to produce virtually any desired peptide. The discardable sequence is manipulated according to the invention in part to have arginine as its carboxyl end. The arginine is in turn coupled by its peptide bond to the N-terminus of the desired peptide. The cleavage of that designated arginine according to the invention is so selective that the desired peptide may contain essentially any sequence of amino acids. The cleavage produces a single copy of the desired peptide.

Although it is not to be regarded as a limitation of the invention, the selectivity of this enzymatic cleavage is believed to be the result of the influence of secondary binding sites of the substrate with the enzyme, clostripain. These secondary sites are adjacent to the primary cleavage site and are known as the P and P′ sites. There may be one or multiple P and P′ sites. The P sites align with the amino acid residues on the amino side of the scissile bond while the P′ sites align with the amino acid residues on the carboxyl side of the scissile bond. Thus, the scissile bond resides between the P and the P′ bond. The corresponding sites of the enzyme are termed S and S′ sites. It is believed that the side chain character of the P and P′ amino acid residues immediately adjacent the primary cleavage residue have significant influence upon the ability of the enzyme to bind with and cleave the peptide bond at the primary cleavage site.

For clostripain, it has been discovered that an acidic amino acid residue occupying the P₂ site (amino side) immediately adjacent to the P₁ primary cleavage amino acid residue, arginine, causes highly selective, rapid attack of clostripain upon that particular primary cleavage site. It has also been discovered that an acidic amino acid residue occupying the P₁′ site (carboxyl side) immediately adjacent the primary cleavage site causes repulsion of, and extremely slow attack of, clostripain upon the-primary cleavage site.

Thus, according to a preferred method of the invention, a polypeptide that has at least one copy of a peptide of interest may be recombinantly produced. The production may be of a soluble polypeptide or, as described in the copending applications filed on even date herewith and having attorney docket numbers 1627.009PRV and 1627.010PRV, the disclosures of which are incorporated herein by reference, an inclusion body preparation containing at least a substantially insoluble-mass of polypeptide. Next, the polypeptide is proteolytically cleaved using clostripain to produce the peptide of interest. By manipulating the polypeptide and/or the cleavage conditions, peptides having any C-terminal residue can be produced. Further, by use of the method of the present invention or by combining the method of this invention with others known in the art, peptides having any C-terminal residue amide can be produced For example GLP-1(7-36)-NH₂ and GLP-1(7-37) can be produced through use of the methods disclosed in this application.

### THE CLOSTRIPAIN CLEAVAGE PROCESS, ACCORDING TO THE INVENTION

According to the invention, clostripain is used in a selective manner to affect preferential cleavage at a selected arginine site. As explained below, clostripain is recognized to cleave at the carboxyl side of arginine and lysine residues in peptides. One of the surprising features of the present invention is the discovery of the ability to provide a selective cleavage site for clostripain so that it will preferentially cleave at a designated arginine even though other arginine or lysine residues are present within the peptide. Multicopy polypeptides having arginine residues at the inchoate C-termini of the desired peptide product copies within the polypeptide and also having arginine or lysine residues within the desired peptide sequence can be efficiently and selectively cleaved according to the invention to produce the desired peptide product.

Moreover, the enzymatic cleavage, precursor polypeptide and desired peptide product can be manipulated so that the C-terminus of the peptide product may be any amino acid residue. This feature is surprising in view of the cleavage preference of clostripain toward arginine. This feature is accomplished through use of a discardable sequence ending in arginine and joined to the N-terminus of the desired peptide. The cleavage of that designated arginine according to the invention is so selective that the desired peptide may contain essentially any sequence of amino acids. The cleavage produces a single copy of the desired peptide.

Traditional Clostripain Cleavage Conditions

Clostripain (EC 3.4.22.8) is an extracellular protease from Clostridia that can be recovered from the culture filtrate of Clostridizini histolyticum. Clostripain has both proteolytic and amidase/esterase activity. (Mitchell et al., Biol. Chem., 243 (18): 4683 (1968)). Clostripain is a heterodimer with a molecular weight of about 50,000 and an isoelectric point of pH 4.8 to 4.9. Clostripain proteolytic activity is inhibited, for example, by tosyl-L-lysine chloromethyl ketone, hydrogen peroxide, Co⁺⁺, Cu⁺⁺ or Cd⁺⁺ ions, citrate, or chelators, such as EGTA and EDTA that bind Ca⁺⁺. Examples of clostripain activators include cysteine, mercaptoethanol, dithiothreitol and calcium ions.

Clostripain is generally understood to have specificity for cleavage of Arg-Xaa linkages, though some cleavage can occur at lysine residues under certain reaction conditions. Thus, in the isolated B chain of insulin, clostripain cleaves the Arg-Gly linkage 500 times more rapidly than the Lys-Ala linkage. In glucagon, only the Arg-Arg, the Arg-Ala and the Lys-Tyr sites are cleaved. The relative initial rates of hydrolysis of these three bonds are 1, 1/7 and 1/300. (Labouesses B., Bull. Soc. Chim. Biol., 42, 1293, 1960).

### CLOSTRIPAIN CLEAVAGE ACCORDING TO THE INVENTION

According to the invention, amino acids flanking arginine can strongly influence clostripain cleavage. In particular, clostripain has a strong preference for a polypeptide having a cleavage site of Formula I, where the cleavage occurs at a peptide bond after amino acid Xaa₂:
Xaa₁-Xaa₂-Xaa₃ (I)
wherein
Xaa₁ aspartic acid, glycine, proline or glutamic acid;
Xaa₂ is arginine; and
Xaa₃ is not an acidic amino acid.

According to the method of the invention, a polypeptide that has at least one copy of a desired peptide first is recombinantly produced. The production may be of a soluble polypeptide or may be an inclusion body preparation containing at least a substantially insoluble mass of polypeptide. Next, the polypeptide is proteolytically cleaved using clostripain to produce the desired peptide. The proteolytic reaction can be performed on the solublized cellular contents in situations where the polypeptide is soluble. Or, it may be performed on crude preparations of inclusion bodies. In either situation, separation steps prior to or following the enzymatic cleavage may be employed. Use of varying concentrations of urea in the medium containing the crude cellular contents or inclusion bodies in optional combination with such separation steps may also be employed. A reaction vessel can also be used that permits continuous recovery and separation of the peptide away from the uncleaved polypeptide and the clostripain. Use of such a method produces large amounts of pure peptide in essentially one step, eliminating numerous processing steps typically used in currently available procedures.

Clostripain can be used to cleave purified or impure preparations of the polypeptide. The precursor polypeptide can be in solution or it can be an insoluble mass. For example, the precursor polypeptide can be in a preparation of inclusion bodies that becomes soluble in the reaction mixture. According to the invention, clostripain is active in high levels of reagents that are commonly used to solubilize proteins. For example, clostripain is active in high levels of urea. Therefore, concentrations of urea ranging up to about 8 M can readily be used in the clostripain cleavage reaction.

Little purification of the polypeptide is required when an inclusion body preparation of the polypeptide is used as a substrate for clostripain cleavage. Essentially, host cells having a recombinant nucleic acid encoding the polypeptide are grown under conditions that permit expression of the polypeptide. Cells are grown to high cell densities, then collected, washed and broken open, for example, by sonication. Inclusion bodies are then collected, washed in water and employed without further purification.

Up to about 8 M urea can be used to solubilized insoluble precursor polypeptides, for example, inclusion body preparations of precursor polypeptides. The amount of urea employed can vary depending on the precursor polypeptide. For example, about 0 M to about 8 M urea can be employed in the clostripain reaction mixture to solubilized the precursor polypeptide. Preferred concentrations of urea are about 4 M urea to about 8 M urea.

Urea can also be used in the clostripain reaction. Concentrations of up to 8 M urea can be used in the clostripain cleavage. Preferred concentrations of urea are about 0.0 to about 4 M urea. More preferred concentrations of urea are about 0.0 to about 1.0 M urea. Even more preferred concentrations of urea are about 0.0 to about 0.5 M urea.

In some cases, it may be preferable to remove the urea before cleavage with clostripain. In such cases, urea may be removed by dialysis, gel filtration, tangential flow filtration (TFF), a multiplicity of chromatographic procedures, and the like.

Moreover, according to the invention, the cleavage reaction conditions can be modified so that clostripain will have an even stronger preference for cleavage at sites having Formula I. Several factors can be modified or implemented to obtain the desired product. Thus, by adjusting the pH or organic solvents, such as ethanol or:acetonitrile, or by using a selected amount of enzyme relative to precursor polypeptide and/or by using selected reaction times and/or by continuously removing the peptide as it is formed, or any combination of the foregoing cleavage at undesired sites can be avoided.

Appropriate inorganic or organic buffers can be used to control the pH of the cleavage reaction. Such buffers include phosphate, Tris, glycine, HEPES and the like. The pH of the reaction can vary between pH 4 and pH 12. However, a pH range between pH 6 and pH 10 is preferred. For amidation, a pH range between 8.5 and 10.5 is preferred. While for hydrolysis, a pH range between 6 and 7 is preferred. When the cleavage is performed on precursor polypeptides in the absence or presence of significant amounts of urea, pH values ranging from about 6.0 to about 6.9 are preferred.

The activity of the clostripain enzyme has surprisingly been found to be influenced by the presence of organic solvents. For example, ethanol and acetonitrile can increase the rate of substrate cleavage as well as the overall yield of product formed from the cleavage of a precursor polypeptide. Another surprising,result is that organic solvents influence the cleavage specificity of clostripain. Thus, an organic solvent can be used to dramatically influence the preferential hydrolysis of one cleavage site in a precursor polypeptide relative to another cleavage site within the same precursor polypeptide. This characteristic of clostripain can be exploited to design precursor polypeptides that are rapidly and preferentially cleaved at specific sites within the precursor polypeptide.

The clostripain enzyme can be activated at similar pH ranges. A suitable buffer substance, for example phosphate, Tris, HEPES, glycine and the like, can be added to maintain the pH.

The concentration of the precursor polypeptide employed during the cleavage is, for example, between 0.01 mg/ml and 100 mg/ml, preferably between 0.1 mg/ml and 20 mg/ml. The preferred ratio of polypeptide to clostripain is, in mg to units, about 1:0.01 to about 1:1,000, more preferably about 1:0.1 to about 1:50.

The temperature of the reaction can also be varied over a wide range and may depend upon the selected reaction conditions. Such a range can be between 0° C. and +80° C. A preferred temperature range is generally between +5° C. and +60° C. Amidation is preferably conducted at a temperature between 5° C. and 60° C., and is more preferably conducted at a temperature between 35° C. and 60° C., and is most preferably conducted at 45° C. Hydrolysis is preferably conducted at a temperature between 20° C. and 30° C., and more preferably is conducted at 25° C.

The time required for the conversion of the precursor polypeptides into the peptides of interest can vary and one of skill in the art can readily ascertain an appropriate reaction time. For example, the reaction time can vary between about 1 min and 48 h. However, a reaction time of between 0.5 h and 6 h is preferred. A reaction time of 0.5 h and 2 hours is more preferred. In some embodiments, the reaction mixture is preferably placed in a reaction vessel that permits continuous removal of the peptide product. For example, the reaction vessel can have a filter that permits the peptide product of interest to pass through but that retains the precursor polypeptide and the clostripain. An example of an appropriate filtration system is tangential flow filtration (TFF). Reaction buffer, substrate and other components of the reaction mixture can be added batchwise or continuously as the peptide is removed and the reaction volume is lost.

The enzyme can be activated before use in a suitable manner in the presence of a mercaptan. Mercaptans suitable for activation are compounds containing SH groups. Examples of such activating compounds include DTT, DTE, mercaptoethanol, thioglycolic acid or cysteine. Cysteine is preferably used. The concentration of the mercaptan can also vary. In general, concentrations between about 0.01 mM and 50 mM are useful. Preferred mercaptan concentrations include concentrations between about 0.05 mM and 5 mM. More preferred mercaptan concentrations are between about 0.5 mM and 2 mM. The activation temperature can be between 0° C. and 80° C. Preferably the activation temperature can be between 0° C. and 40° C., more preferably the activation temperature is between 0° C. and 30° C. Most preferably the activation temperature is between 15° C. and 25° C.

Clostripain can be purchased from commercially available sources or it can be isolated from microorganisms. Natural and recombinant clostripain is available. For example, natural clostripain can be prepared from Clostridia bacteria by cultivating the bacteria until clostripain accumulates in the nutrient medium. Clostridia used for producing clostripain include, for example, Clostridium histolyticum, especially Clostridium histolyticum DSM 627. Culturing is carried out anaerobically, singly or in mixed culture, for example, in non-agitated culture in the absence of oxygen or in fermentors. Where appropriate, nitrogen, inert gases or other gases apart from oxygen can be introduced into the culture. The fermentation is carried out in a temperature range from about 10° to 45° C., preferably about 25° to 40° C., especially 30° to 38° C. Fermentation takes place in a pH range between 5 and 8.5, preferably between 5.5 and 8. Under these conditions, the culture broth generally shows a detectable accumulation of the enzyme after 1 to 3 days. The synthesis of clostripain starts in the late log phase and reaches its maximum in the stationary phase of growth. The production of the enzyme can be followed by means of activity assays (Mitchell, Meth. of Enzymol. 47: 165 (1977)).

The nutrient solution used for producing clostripain can contain 0.2 to 6%, preferably 0.5 to 3%, of organic nitrogen compounds and inorganic salts. Suitable organic nitrogen compounds are: amino acids, peptones, meat extracts, milled seeds, for example of corn, wheat, beans, soybean or the cotton plant, distillation residues from alcohol production, meat meals or yeast extracts. Examples of inorganic salts that the nutrient solution can contain are chlorides, carbonates, sulfates or phosphates of the alkali metals or alkaline earth metals, iron, zinc and manganese, also ammonium salts and nitrates.

Clostripain can be purified by classical processes, for example by ammonium sulfate precipitation, ion exchange or gel permeation chromatography or it can be produced recombinantly.

### PEPTIDES OF INTEREST SERVING AS SUBSTRATES ACCORDING TO THE INVENTION

Almost any peptide can be formed by the methods of the invention. Peptides with an arginine at their C-terminus can readily be cleaved from a polypeptide containing end-to-end copies of the peptide. Peptides with one or more internal arginine residues can also be made by employing the teachings of the invention on which arginine-containing sites are favored for cleavage. Peptides having C-terminal amino acids other than arginine can be produced by placing a clostripain cleavage site within the polypeptide at the N-terminus of the peptide of interest. This latter technique produces the single copy desired peptide and employs a recombinantly expressed polypeptide having a discardable peptide sequence at the N-terminal side of the desired peptide.

Clostripain is generally perceived to be an “arginine” or an “arginine/lysine” protease, meaning that clostripain cleaves polypeptides on the carboxyl side of arginine and/or lysine amino acid residues. However, according to the invention, clostripain has even greater specificity, particularly under the reaction conditions provided herein. Hence, peptides with internal lysine and arginine residues can be made by the procedures of the invention.

Moreover, the construction of the polypeptide can be manipulated so that the peptide of interest is present at the C-terminus of the polypeptide and a clostripain cleavage site is at the N-terminus of the peptide of interest. Hence, when cleavage is performed on a polypeptide containing such a C-terminal peptide, the peptide is readily released. Using such a precursor polypeptide, peptides with any C-terminal residue can be formed.

According to the invention, peptides having one or more internal arginine residues can still be selectively cleaved at their termini so that a functional, full-length peptide can be recovered. This enhanced selectivity is achieved by recognition that clostripain preferentially cleaves a polypeptide having a site sequence-given by Formula I, where the cleavage occurs at a peptide bond after amino acid Xaa₂:
Xaa₁-Xaa₂-Xaa₃ (I)
wherein
Xaa₁ aspartic acid, glycine, proline or glutamic acid;
Xaa₂ is arginine; and
Xaa₃ is not an acidic amino acid.

Hence, a peptide of the Formula Xaa₃-Peptide₁-Xaa₁-Xaa₂, can readily be excised from a polypeptide having end-to-end concatemers of the peptide, when Xaa₁, Xaa₂, and Xaa₃ are as described above. Peptide1 refers to a peptidyl entity that is unique to the selected peptide of interest. Hence, Peptide₁ has any amino acid sequence that is selected by one of skill in the art. An example of such a polypeptide with end-to-end concatemers of the peptide of interest has Formula II:
(Xaa₃-Peptide₁-Xaa₁-Xaa₂)ₙ-Xaa₃-Peptide₁-Xaa₁-Xaa₂ (II)
wherein
the peptide comprises Xaa₃-Peptide₁-Xaa₁-Xaa₂;
n is an integer ranging from 0 to 50;
Xaa₁ is aspartic acid, glycine, proline or glutamic acid;
Xaa₂ is arginine; and
Xaa₃ is not an acidic amino acid.

However, the invention is not limited to cleavage of polypeptides having end-to-end concatemers of a peptide of interest. The invention also provides methods of making large amounts of a peptide that is present as a single copy within a polypeptide. This aspect of the invention enables the production of a single copy desired peptide having virtually any amino acid sequence and one not having an arginine at the C-terminus. That is, the invention provides methods of making large amounts of peptides of the Formula Xaa₃-Peptide₁, which do not have a C-terminal lysine or arginine. A cleavable peptide linker can be attached onto the peptide (e.g. Linker-Xaa₃-Peptide₁) to generate an N-terminal cleavage site for generating peptides of interest that have no C-terminal arginine or lysine. The Linker has a C-terminal Xaa₁-Xaa₂ sequence that directs cleavage to the junction between the C-terminal Xaa₂ residue of the Linker and the Xaa₃ N-terminal residue of the peptide. Hence, peptides of the Formula Xaa₃-Peptide₁ that have C-terminal acidic, aliphatic or aromatic amino acids can readily be made by the present methods.

Cleavage of a peptide of the Formula Xaa₃-Peptide₁ from a polypeptide having at least one copy of the peptide relies upon the presence of a site that has Formula I (Xaa₁-Xaa₂-Xaa₃) at the junction between the peptide and the attached Linker or polypeptide. The Xaa₃ amino acid forms the N-terminal end of the peptide and is not an acidic amino acid. Sequence. Polypeptides of Formula III can readily be cleaved by clostripain:
(Linker-Xaa₁-Xaa₂-Xaa₃-Peptide₁)ₙ-Linker-Xaa₁-Xaa₂-Xaa₃-Peptide₁ Formula III
wherein
the peptide comprises Xaa₃-Peptide₁
n is an integer ranging from 0 to 50;
Xaa₁ is aspartic acid, glycine, proline or glutamic acid;
Xaa₂ is arginine; and
Xaa₃ is not an acidic amino acid.

Cleavage of a polypeptide of Formula III yields one molar equivalent of the Xaa₃-Peptide₁ and n molar equivalents of a polypeptide of the following structure: Xaa₃-Peptide₁-Linker-Xaa₁-Xaa₂. While this polypeptide may not have a specific utility after cleavage, many “unused” parts of the linker or the polypeptide do have specific purposes. For example, the Xaa₁-Xaa₂ amino acids in the polypeptide are recognized by and direct clostripain to cleave the Xaa₂-Xaa₃ peptide bond with specificity. As described in the section entitled “Precursor polypeptides,” other parts of the polypeptide or the linker have specific functions relating to the recombinant expression, translation, sub-cellular localization, etc. of the polypeptide within the host cell.

Almost any peptide of interest to one of skill in the art can be made by the methods of the invention. In particular, preferred peptides of interest (desired peptides) include, for example, a GLP-1 (7-36) glucagon-like peptide. Types of GLPs that can be made by the methods of the invention include, for example, GLP-1(7-36) (SEQ ID NO:1), GLP-1 (7-36)amide (SEQ ID NO:2), GLP-1 (7-37) (SEQ ID NO:3), GLP-1 (7-37)amide (SEQ ID NO:4), GLP-1 (7-36) K26R (SEQ ID NO:5), GLP-1(7-36) K26R—NH₂ (SEQ ID NO:6), GLP-1 (7-37) K26R (SEQ ID NO:7), GLP-1(7-37) K26R—NH₂ (SEQ ID NO:8), as well as conservative amino acid substitutions thereof. The sequences of such GLPs are provided in Table 1 along with their names and SEQ ID NO: (“NO:”).

TABLE 1

| | | |
|---|---|---|
| Name | Sequence | NO: |

| | | |
|---|---|---|
| GLP-1(7-36) | HAEGTFTSDVSSYLEGQAAKEFIA | 1 |
| | WLVKGR | |
| GLP-1(7-36) NH₂ | HAEGTFTSDVSSYLEGQAAKEFIA | 2 |
| | WLVKGR-NH₂ | |
| GLP-1(7-37) | HAEGTFTSDVSSYLEGQAAKEFIA | 3 |
| | WLVKGRG | |
| GLP-1(7-37) NH₂ | HAEGTFTSDVSSYLEGQAAKEFIA | 4 |
| | WLVKGRG-NH₂ | |
| GLP-1(7-36) K26R | HAEGTFTSDVSSYLEGQAAREFIA | 5 |
| | WLVKGR | |
| GLP-1(7-36) K26R- | HAEGTFTSDVSSYLEGQAAREFIA | 6 |
| NH₂ | WLVKGR-NH₂ | |
| GLP-1(7-37) K26R | HAEGTFTSDVSSYLEGQAAREFIA | 7 |
| | WLVKGRG | |
| GLP-1(7-37) K26R- | HAEGTFTSDVSSYLEGQAAREFIA | 8 |
| NH₂ | WLVKGRG-NH₂ | |

The peptide GLP-1 (7-36) (SEQ ID NO:1) is numbered 7-36 for historical reasons. The original sequencing studies indicated that GLP-1 was the product of a gene that encoded thirty-seven amino acids. However, it was subsequently found that the active peptide did not have residues 1-6, and that the glycine at position 37 was degraded to form an amide at position 36.

The invention also contemplates peptide variants derivatives of the GLP peptides described herein. Derivative and variant peptides of the invention are derived from the reference peptide by deletion or addition of one or more amino acids to the N-terminal and/or C-terminal end; deletion or addition of one or more amino acids at one or more sites within the peptide; or substitution of one or more amino acids at one or more sites peptide. Thus, the GLP-1 peptides of the invention may be altered in various ways including amino acid substitutions, deletions, truncations, and insertions. The invention also includes the GLP-1 peptides, analogs and derivatives disclosed in U.S. Pat. Nos. 5,574,008; 6,133,235 and 6,277,819 the disclosures and peptide formulas of which are incorporated herein by reference.

Such variant and derivative GLP-1 polypeptides may result, for example, from human manipulation. Methods for such manipulations are generally known in the art. For example, amino acid sequence variants of the polypeptides can be prepared by mutations in the DNA. Methods for mutagenesis and nucleotide sequence alterations are well known in the art. See, for example, Kunkel, Proc. Natl. Acad. Sci. USA, 82: 488 (1985); Kunkel et al., Methods in Enzymol., 154: 367 (1987); U.S. Pat. No. 4,873,192; Walker and Gaastra, eds., Techniques in Molecular Biology, MacMillan Publishing Company, New York (1983) and the references cited therein. Guidance as to appropriate amino acid substitutions that do not affect biological activity of the protein of interest may be found in the model of Dayhoff et al., Atlas of Protein Sequence and Structure, Natl. Biomed. Res. Found., Washington, C.D. (1978), herein incorporated by reference.

Precursor Polypeptides

Any precursor polypeptide containing one or more copies of a peptide of interest (desired peptide) and a Formula I sequence at one or both ends of that peptide can be utilized as a substrate for the clostripain cleavage methods of the invention. One of skill in the art can readily design many such precursor polypeptides. While the peptide of interest may form a substantial portion of the precursor polypeptide, the polypeptide may also have additional peptide segments unrelated to the peptide sequence of interest. Additional peptide segments can provide any function desired by one of skill in the art.

One example of an additional peptide segment that can be present in the precursor polypeptide is a “Tag” that provides greater levels of precursor polypeptide production in cells. Numerous tag sequences are known in the art. In the present invention, bacterial tag sequences are preferred. Such tag sequences may be obtained from gene 10 of bacteriophage T7, and the gene encoding ompT. In one embodiment, a T7 tag is used that has the amino acid sequence, ASMTGGQQMGR (SEQ ID NO:17), or MASMTGGQQMGR (SEQ ID NO:18).

The precursor polypeptide can also encode an “inclusion body leader partner” that is operably linked to the peptide of interest. Such an inclusion body leader partner may be linked to the amino-terminus, the carboxyl-terminus or both termini of a precursor polypeptide. In one example, the inclusion body leader partner has an amino acid sequence corresponding to: GSGQGQAQYLSASCVVFTYSGDTASQVD (SEQ ID NO:19). In another embodiment, the inclusion body leader partner is a part of the Drosophila vestigial polypeptide (“Vg”), having sequence GSGQGQAQYLAASLVVF TNYSGDTASQ VDVNGPRAMVD (SEQ ID NO:20). In another embodiment, the inclusion body leader partner is a part of polyhedrin polypeptide (“Ph”), having sequence GSAEEEEILLEVSLVFKVKEFAPDAPLFTGPAYVD (SEQ ID NO:21). Other inclusion body leader partners that can be used include a part of the lactamase polypeptide, having sequence SIQHFRVALIPFFAAFSLPVFA (SEQ ID NO:22). Upon expression of the polypeptide, an attached inclusion body leader partner causes the polypeptide to form inclusion bodies within the bacterial host cell. Other inclusion body leader partners can be identified, for example, by linking a test inclusion body leader partner to a polypeptide construct. The resulting inclusion body leader partner-polypeptide construct then would be tested to determine whether it will form an inclusion body within a cell.

The amino acid sequence of an inclusion body leader partner can be altered to produce inclusion bodies that facilitate isolation of inclusion bodies that are formed, thereby allowing an attached polypeptide to be purified more easily. For example, the inclusion body leader partner may be altered to produce inclusion bodies that are more or less soluble under a certain set of conditions. Those of skill in the art realize that solubility is dependent on a number of variables that include, but are not limited to, pH, temperature, salt concentration, protein concentration and the hydrophilicity or hydrophobicity of the amino acids in the protein. Thus, an inclusion body leader partner of the invention may be altered to produce an inclusion body having desired solubility under differing conditions.

An inclusion body leader partner may also be altered to produce inclusion bodies that contain polypeptide constructs having greater or lesser self-association. Self-association refers to the strength of the interaction between two or more polypeptides that form an inclusion body. Such self-association may be determined though use of a variety of known methods used to measure protein-protein interactions. Such methods are known in the art and have been described (Freifelder, Physical Biochemistry: Applications to Biochemistry and Molecular Biology, W.H. Freeman and Co., 2nd edition, New York, N.Y. (1982)).

Self-adhesion can be used to produce inclusion bodies that exhibit varying stability to purification. For example, greater self-adhesion may be desirable to stabilize inclusion bodies against dissociation in instances where harsh conditions are used to isolate the inclusion bodies from a cell. Such conditions may be encountered if inclusion bodies are being isolated from cells having thick cell walls. However, where mild conditions are used to isolate the inclusion bodies, less self-adhesion may be desirable as it may allow the polypeptide constructs composing the inclusion body to be more readily solubilized or processed. Accordingly, an inclusion body leader partner of the invention may be altered to provide a desired level of self-adhesion for a given set of conditions.

The precursor polypeptide can also encode one or more “cleavable peptide linkers” that can flank one or more copies of the peptide of interest. Such a cleavable peptide linker provides a convenient clostripain cleavage site adjacent to a peptide of interest, and allows a peptide that does not naturally begin or end with an arginine or lysine to be excised with clostripain. Convenient cleavable peptide linkers include short peptidyl sequences having a C-terminal Xaa₁-Xaa₂ sequence, for example, a Linker-Xaa₁-Xaa₂ sequence, wherein Xaa₁ is aspartic acid, glycine, proline or glutamic acid, and Xaa₂ is arginine. The Xaa₁-Xaa₂ sequence directs cleavage to the junction between the C-terminal Xaa₂ residue of the linker and a Xaa₃ residue on the N-terminus of the peptide.

A cleavable peptide linker can have the following Formula IV:
(Peptide₅)ₘ-Xaa₁-Xaa₂ IV
wherein:
n and m are separately an integer ranging from 0 to 50;
Xaa₁ is aspartic acid, glycine, proline or glutamic acid; and
Xaa₂ is arginine; and
Peptide₅ is any single or multiple amino acid residue.
   In some embodiments, use of either Xaa₄ or Xaa₅ as proline is preferred.

Many cleavable peptide linker sequences can readily be developed and used by one of skill in the art. A few examples of convenient cleavable peptide linker sequences are provided below.
Ala-Phe-Leu-Gly-Pro-Gly-Asp-Arg (SEQ ID NO:23)
Val-Asp-Asp-Arg.(SEQ ID NO:24)
Gly-Ser-Asp-Arg (SEQ ID NO:25)
Ile-Thr-Asp-Arg (SEQ ED NO:26)
Pro-Gly-Asp-Arg (SEQ ID NO:27).

Other amino acids, peptides, or polypeptides selected by one of skill in the art can also be included in the precursor polypeptide.

GLP-1 Polypeptides

In another embodiment of the invention, the polypeptide can encode one or more copies of GLP-1. An example of a polypeptide encoding one copy of GLP-1 is a polypeptide having the following generalized structure:
Tag-Linker-[GLP-1(7-36)-Linker₂]_{q} VII
wherein Linker is a described above. Preferably, Linker is Linker₁, defined herein as Peptide₅-Asp-Arg. The variable q is an integer of about 2 to about 20. A preferred value for q in this case is 3. As provided above, the nucleic acid encoding the Peptide₅ amino acids can provide convenient restriction sites for cloning purposes so long as an amino acid codon (rather than, for example, a stop codon) is still encoded by the nucleic acid. While any appropriate sequence can be used for Peptide₅, a preferred sequence is Ile-Thr.

Linker₂ is a cleavable peptide linker having the sequence AFLGPGDR (SEQ ID NO:23). A multi-copy GLP-1(7-36) (SEQ ID NO:1) polypeptide of this generalized structure with q equal to 3 has the following sequence:

(SEQ ID NO:31)

ASMTGGQQMGRGS-Peptide₅-Asp-Arg-
HAEGTFTSDVSSYLEGQAAKEFIAWLVKGR-AFLGPGDR
HAEGTFTSDVSSYLEGQAAKEFIAWLVKGR-AFLGPGDR
HAEGTFTSDVSSYLEGQAAKEFIAWLVKGR

One mutation that can be made is a substitution of arginine for lysine at position 26 of the GLP-1 peptide, to produce GLP-1(7-36, K26R) having SEQ ID NO:5 or GLP-1(7-37, K26R) having SEQ ID NO:7. This amino acid substitution of arginine for lysine produces a GLP-1 peptide with one lysine at position 34. In some embodiments, one of skill in the art may choose to derivatize the lysine at position 34, in which case having an arginine at position 26 eliminates the potential for derivatization at two sites.
Amidation Conditions

When clipped from a multicopy polypeptide under normal hydrolysis conditions, recombinant GLP-1 has a C terminal carboxyl group. However, an amidated C-terminus is preferred for use in mammals. Clostripain can be used to amidate the C-terminal residue to make an amidated recombinant GLP-1 by adjusting the conditions to increase the amount of amide formation. However, the recombinant GLP-1 amide itself becomes a substrate for hydrolysis as it is formed. To solve this problem, a tangential flow filtration in combination with the enzyme reaction may be used. Clostripain simultaneously cleaves multicopy peptide constructs and amidates the C-terminal residue of the single copy cleaved peptide. Use of tangential flow filtration during the enzymatic reaction to remove the amidated peptide produces that peptide in high yield.

For example, use of a 10K diafiltration/tangential flow filtration membrane will enhance the reaction yield. Undigested peptide-construct and clostripain are retained on the retentate side of the membrane. The single copy cleaved rGLP-1 passes through the membrane. Continued exposure of rGLP-1 amide to clostripain will result in loss of the amide to OH. Continual removal of amide through the membrane will reduce this unwanted side reaction. Smaller pore sized membranes were not as efficient at removing the newly formed RGLP-1 amide during the reaction time course.

Clostripain, like other proteases, will perform transpeptidation reactions in the presence of a nucleophile other than water. Ammonia or other amines can be used as the nucleophile. A polypeptide that had three copies of the GLP-1 peptide was used as a substrate. The polypeptide had a leader sequence as well.

Reaction conditions will enhance the transpeptidation reaction relative to hydrolysis for this particular polypeptide construct. Urea in the clostripain reaction maintains peptide solubility and minimizes membrane fouling. The clostripain digestion/amidation reaction will tolerate higher urea concentrations. The amount of clostripain can be varied to shorten or lengthen the overall reaction time. Fresh buffer can be added to maintain constant volume or after volume reduction.

Production of Precursor Polypeptides

A) DNA Constructs and Expression Cassettes

Precursor polypeptides are produced in any convenient manner, for example, by using a recombinant nucleic acid that encodes the desired precursor polypeptide. Nucleic acids encoding the precursor polypeptides of the invention can be inserted into convenient vectors for transformation of an appropriate host cell. Those of skill in the art can readily obtain and clone nucleic acids encoding a selected precursor polypeptide into a variety of commercially available plasmids. One example of a useful plasmid vector is the pET series of plasmids (Stratagene, La Jolla, Calif.). After insertion of the selected nucleic acid into an appropriate vector, the vector can be introduced into a host cell, preferably a bacterial host cell.

Nucleic acid constructs and expression cassettes can be created through use of recombinant methods that are available in the art. (Sambrook and Russell, Molecular Cloning: A Laboratory Manual, 3rd edition (Jan. 15, 2001) Cold Spring Harbor Laboratory Press, ISBN: 0879695765; Ausubel et al., Current Protocols in Molecular Biology, Green Publishing Associates and Wiley Interscience, NY (1989)). Generally, recombinant methods involve preparation of a desired DNA fragment and ligation of that DNA fragment into a preselected position in another DNA vector, such as a plasmid.

In a typical example, a desired DNA fragment is first obtained by synthesizing and/or digesting a DNA that contains the desired DNA fragment with one or more restriction enzymes that cut on both sides of the desired DNA fragment. The restriction enzymes may leave a “blunt” end or a “sticky” end. A “blunt” end means that the end of a DNA fragment does not contain a region of single-stranded DNA. A DNA fragment having a “sticky” end means that the end of the DNA fragment has a region of single-stranded DNA. The sticky end may have a 5′ or a 3′ overhang. Numerous restriction enzymes are commercially available and conditions for their use are also well known. (USB, Cleveland, Ohio; New England Biolabs, Beverly, Mass.).

The digested DNA fragments may be extracted according to known methods, such as phenol/chloroform extraction, to produce DNA fragments free from restriction enzymes. The restriction enzymes may also be inactivated with heat or other suitable means. Alternatively, a desired DNA fragment may be isolated away from additional nucleic acid sequences and restriction enzymes through use of electrophoresis, such as agarose gel or polyacrylamide gel electrophoresis. Generally, agarose gel electrophoresis is used to isolate large nucleic acid fragments while polyacrylamide gel electrophoresis is used to isolate small nucleic acid fragments. Such methods are used routinely to isolate DNA fragments. The electrophoresed DNA fragment can then be extracted from the gel following electrophoresis through use of many known methods, such as electroelution, column chromatography, or binding of glass beads. Many kits containing materials and methods for extraction and isolation of DNA fragments are commercially available. (Qiagen, Venlo, Netherlands; Qbiogene, Carlsbad, Calif.).

The DNA segment into which the fragment is going to be inserted is then digested with one or more restriction enzymes. Preferably, the DNA segment is digested with the same restriction enzymes used to produce the desired DNA fragment. This will allow for directional insertion of the DNA fragment into the DNA segment based on the orientation of the complimentary ends. For example, if a DNA fragment is produced that has: an EcoRI site on its 5′ end and a BamHI site at the 3′ end, it may be directionally inserted into a DNA segment that has been digested with EcoRI and BamHI based on the complementarity of the ends of the respective DNAs. Alternatively, blunt ended cloning may be used if no convenient restriction sites exist that allow for directional cloning. For example, the restriction enzyme BsaAI leaves DNA ends that do not have a 5′ or 3′ overhang. Blunt ended cloning may be used to insert a DNA fragment into a DNA segment that was also digested with an enzyme that produces a blunt end. Additionally, DNA fragments and segments may be digested with a restriction enzyme that produces an overhang and then treated with an appropriate enzyme to produce a blunt end. Such enzymes include polymerases and exonucleases. Those of skill in the art know how to use such methods alone or in combination to selectively produce DNA fragments and segments that may be selectively combined.

A DNA fragment and a DNA segment can be combined though conducting a ligation reaction. Ligation links two pieces of DNA through formation of a phosphodiester bond between the two pieces of DNA. Generally, ligation of two or more pieces of DNA occurs through the action of the enzyme ligase when the pieces of DNA are incubated with ligase under appropriate conditions. Ligase and methods and conditions for its use are well known in the art and are commercially available.

The ligation reaction or a portion thereof is then used to transform cells to amplify the recombinant DNA formed, such as a plasmid having an insert. Methods for introducing DNA into cells are well known and are disclosed herein.

Those of skill in the art recognize that many techniques for producing recombinant nucleic acids can be used to produce an expression cassette or nucleic acid construct of the invention.

B) Promoters

The expression cassette of the invention includes a promoter. Any promoter able to direct transcription of the expression cassette may be used. Accordingly, many promoters may be included within the expression cassette of the invention. Some useful promoters include, constitutive promoters, inducible promoters, regulated promoters, cell specific promoters, viral promoters, and synthetic promoters. A promoter is a nucleotide sequence which controls expression of an operably linked nucleic acid sequence by providing a recognition site for RNA polymerase, and possibly other factors, required for proper transcription. A promoter includes a minimal promoter, consisting only of all basal elements needed for transcription initiation, such as a TATA-box and/or other sequences that serve to specify, the site of transcription initiation. A promoter may be obtained from a variety of different sources. For example, a promoter may be derived entirely from a native gene, be composed of different elements derived from different promoters found in nature, or be composed of nucleic acid sequences that are entirely synthetic. A promoter may be derived from many different types of organisms and tailored for use within a given cell.

Examples of Promoters Useful in Bacteria

For expression of a leader protein in a bacterium, an expression cassette having a bacterial promoter will be used. A bacterial promoter is any DNA sequence capable of binding bacterial RNA polymerase and initiating the downstream (3″) transcription of a coding sequence into mRNA. A promoter will have a transcription initiation region which is usually placed proximal to the 5′ end of the coding sequence. This transcription initiation region usually includes an RNA polymerase binding site and a transcription initiation site. A second domain called an operator may be present and overlap an adjacent RNA polymerase binding site at which RNA synthesis begins. The operator permits negatively regulated (inducible) transcription, as a gene repressor protein may bind the operator and thereby inhibit transcription of a specific gene. Constitutive expression may occur in the absence of negative regulatory elements, such as the operator. In addition, positive regulation maybe achieved by a gene activator protein binding sequence, which, if present is usually proximal (5′) to the RNA polymerase binding sequence. An example of a gene activator protein is the catabolite activator protein: (CAP), which helps initiate transcription of the lac operon in E. coli (Raibaud et al., Ann. Rev. Genet., 18:173 (1984)). Regulated expression may therefore be positive or negative, thereby either enhancing or reducing transcription. A preferred promoter is the Chlorella virus promoter. (U.S. Pat. No. 6,316,224).

Sequences encoding metabolic pathway enzymes provide particularly useful promoter sequences. Examples include promoter sequences derived from sugar metabolizing enzymes, such as galactose, lactose (lac) (Chang et al., Nature, 198:1056 (1977), and maltose. Additional examples include promoter sequences derived from biosynthetic enzymes such as tryptophan (trp) (Goeddel et al., Nuc. Acids Res., 8:4057 (1980); Yelverton et al., Nuc. Acids Res., 9:731 (1981); U.S. Pat. No. 4,738,921; and EPO Publ. Nos. 036 776 and 121 775). The β-lactamase (bla) promoter system (Weissmann, “The cloning of interferon and other mistakes”, in: Interferon 3 (ed. I. Gresser), 1981), and bacteriophage lambda P_{L} (Shimatake et al., Nature 292:128 (1981)) and T5 (U.S. Pat. No. 4,689,406) promoter systems also provide useful promoter sequences.

Synthetic promoters which do not occur in nature also function as bacterial promoters. For example, transcription activation sequences of one bacterial or bacteriophage promoter may be joined with the operon sequences of another bacterial or bacteriophage promoter, creating a synthetic hybrid promoter (U.S. Pat. No. 4,551,433). For example, the tac promoter is a hybrid trp-lac promoter comprised of both trp promoter and lac operon sequences that is regulated by the lac repressor (Amann et al., Gene, 25:167 (1983); de Boer et al., Proc. Natl. Acad. Sci. USA, 80:21 (1983)). Furthermore, a bacterial promoter can include naturally occurring promoters of non-bacterial origin that have the ability to bind bacterial RNA polymerase and initiate transcription. A naturally occurring promoter of non-bacterial origin can also be coupled with a compatible RNA polymerase to produce high levels of expression of some genes in prokaryotes. The bacteriophage T7 RNA polymerase/promoter system is an example of a coupled promoter system (Studier et al., J. Mol. Biol. 189:113 (1986); Tabor et al., Proc. Natl. Acad. Sci. USA, 82:1074 (1985)). In addition, a hybrid promoter can also be comprised of a bacteriophage promoter and an E. coli operator region (EPO Publ. No. 267 851).

Examples of Promoters Useful in Insect Cells

An expression cassette having a baculovirus promoter can be used for expression of a leader protein in an insect cell. A baculovirus promoter is any DNA sequence capable of binding a baculovirus RNA polymerase and initiating transcription of a coding sequence into mRNA. A promoter will have a transcription initiation region which is usually placed proximal to the 5′ end of the coding sequence. This transcription initiation region usually includes an RNA polymerase binding site and a transcription initiation site. A second domain called an enhancer may be present and is usually distal to the structural gene. A baculovirus promoter may be a regulated promoter or a constitutive promoter. Useful promoter sequences may be obtained from structural genes that are transcribed at times late in a viral infection cycle. Examples include sequences derived from the gene encoding the baculoviral polyhedron protein (Friesen et al., “The Regulation of Baculovirus Gene Expression”, in: The Molecular Biology of Baculoviruses (ed. Walter Doerfler), 1986; and EPO Publ. Nos. 127 839 and 155 476) and the gene encoding the baculoviral p10 protein (Vlak et al., J. Gen. Virol., 69:765 (1988)).

Examples of Promoters Useful in Yeast

Promoters that are functional in yeast are known to those of ordinary skill in the art. In addition to an RNA polymerase binding site and a transcription initiation site, a yeast promoter may also have a second region called an upstream activator sequence. The upstream activator sequence permits regulated expression that may be induced. Constitutive expression occurs in the absence of an upstream activator sequence. Regulated expression may be either positive or negative, thereby either enhancing or reducing transcription.

Promoters for use in yeast may be obtained from yeast genes that encode enzymes active in metabolic pathways. Examples of such genes include alcohol dehydrogenase (ADH) (EPO Publ. No. 284 044), enolase, glucokinase, glucose-6-phosphate isomerase, glyceraldehyde-3-phosphatedehydrogenase (GAP or GAPDH), hexokinase, phosphofructokinase, 3-phosphoglyceratemutase, and pyruvate kinase (PyK). (EPO Publ. No. 329 203). The yeast PHO5 gene, encoding acid phosphatase, also provides useful promoter sequences. (Myanohara et al., Proc. Natl. Acad. Sci. USA, 80:1 (1983).

Synthetic promoters which do not occur in nature may also be used for expression in yeast. For example, upstream activator sequences from one yeast promoter may be joined with the transcription activation region of another yeast promoter, creating a synthetic hybrid promoter. Examples of such hybrid promoters include the ADH regulatory sequence linked to the GAP transcription activation region (U.S. Pat. Nos. 4,876,197and 4,880,734). Other examples of hybrid promoters include promoters which consist of the regulatory sequences of either the ADH2, GAL4, GAL10, or PHO5 genes, combined with the transcriptional activation region of a glycolytic enzyme gene such as GAP or PyK (EPO Publ. No. 164 556). Furthermore, a yeast promoter can include naturally occurring promoters of non-yeast origin that have the ability to bind yeast RNA polymerase and initiate transcription. Examples of such promoters are known in the art. (Cohen et al., Proc. Natl. Acad Sci. USA, 77:1078 (1980); Henikoff et al., Nature, 283:835 (1981); Hollenberg et al., Curr. Topics Microbiol. Immunol., 96:119 (1981)); Hollenberg et al., “The Expression of Bacterial Antibiotic Resistance Genes in the Yeast Saccharomyces cerevisiae”, in: Plasmids of Medical, Environmental and Commercial Importance (eds. K. N. Timmis and A. Puhler), 1979; (Mercerau-Puigalon et al., Gene, 11:163 (1980); Panthier et al., Curr. Genet., 2:109 (1980)).

Examples of Promoters Useful in Mammalian Cells

Many mammalian promoters are known in the art that may be used in conjunction with the expression cassette of the invention. Mammalian promoters often have a transcription initiating region, which is, usually placed proximal to the 5′ end of the coding sequence, and a TATA box, usually located 25-30 base pairs (bp) upstream of the transcription initiation site. The TATA box is thought to direct RNA polymerase II to begin RNA synthesis at the correct site. A mammalian promoter may also contain an upstream promoter element, usually located within 100 to 200 bp upstream of the TATA box. An upstream promoter element determines the rate at which transcription is initiated and can act in either orientation (Sambrook et al., “Expression of Cloned Genes in Mammalian Cells”, in: Molecular Cloning: A Laboratory Manual, 2nd ed., 1989).

Mammalian viral genes are often highly expressed and have a broad host range; therefore sequences encoding mammalian viral genes often provide useful promoter sequences. Examples include the SV40 early promoter, mouse mammary tumour virus LTR promoter, adenovirus major late promoter (Ad MLP), and herpes simplex virus promoter. In addition, sequences derived from non-viral genes, such as the murine metallothioneih gene, also provide useful promoter sequences. Expression may be either constitutive or regulated.

A mammalian promoter may also be associated with an enhancer. The presence of an enhancer will usually increase transcription from an associated promoter. An enhancer is a regulatory DNA sequence that can stimulate transcription up to 1000-fold when linked to homologous or heterologous promoters, with synthesis beginning at the normal RNA start site. Enhancers are active when they are placed upstream or downstream from the transcription initiation site, in either normal or flipped orientation, or at a distance of more than 1000 nucleotides from the promoter. (Maniatis et al., Science, 236:1237 (1987)); Alberts et al., Molecular Biology of the Cell, 2nd ed., 1989). Enhancer elements derived from viruses are often times useful, because they usually have a broad host range. Examples include the SV40 early gene enhancer (Dijkema et al., EMBO J. 4:761 (1985)) and the enhancer/promoters derived from the long terminal repeat (LTR) of the Rous Sarcoma Virus (Gorman et al., Proc. Natl. Acad. Sci. USA, 79:6777 (1982b)) and from human cytomegalovirus (Boshart et al., Cell, 41:521 (1-985)). Additionally, some enhancers are regulatable and become active only in the presence of an inducer, such as a hormone or metal ion (Sassone-Corsi and Borelli, Trends Genet., 2:215 (1986); Maniatis et al., Science, 236:1237 (1987)).

It is understood that many promoters and associated regulatory elements maybe used within the expression cassette of the invention to transcribe an encoded leader protein. The promoters described above are provided merely as examples and are not to be considered as a complete list of promoters that are included within the scope of the invention.

C) Translation Initiation Sequence

The expression cassette of the invention may contain a nucleic acid sequence for increasing the translation efficiency of an mRNA encoding a leader protein of the invention. Such increased translation serves to increase production of the leader protein. Tie presence of an efficient ribosome binding site is useful for gene expression in prokaryotes. In bacterial mRNA a conserved stretch of six nucleotides, the Shine-Dalgarno sequence, is usually found upstream of the initiating AUG codon. (Shine et al., Nature. 254:34 (1975)). This sequence is thought to promote ribosome binding to the mRNA by base pairing between the ribosome binding site and the 3′ end of Escherichia coli 16S rRNA (Steitz et al., “Genetic signals and nucleotide sequences in messenger RNA”, in: Biological Regulation and Development: Gene Expression (ed. R. F. Goldberger), 1979)). Such a ribosome binding site, or operable derivatives thereof, are included within the expression cassette of the invention.

A translation initiation sequence can be derived from any expressed Escherichia coli gene and can be used within an expression cassette of the invention. Preferably the gene is a highly expressed gene. A translation initiation sequence can be obtained via standard recombinant methods, synthetic techniques, purification techniques, or combinations thereof, which are all well known. (Ausubel et al., Current Protocols in Molecular Biology, Green Publishing Associates and Wiley Interscience, NY. (1989); Beaucage and Camthers, Tetra Letts., 22:1859 (1981); VanDevanter et al., Nucleic Acids Res., 12:6159 (1984). Alternatively, translational start sequences can be obtained from numerous commercial vendors. (Operon Technologies; Life Technologies Inc, Gaithersburg, Md.). In a preferred embodiment, the T7 leader sequence is used. The T7Tag leader sequence is derive from the highly expressed T7 Gene 10 cistron. Other examples of translation initiation sequences include, but are not limited to, the maltose-binding protein (Mal E gene) start sequence (Guan et al., Gene, 67:21 (1997)) present in the pMalc2 expression vector (New England Biolabs, Beverly, Mass.) and the translation initiation sequence for the following genes: thioredoxin gene (Novagen, Madison, Wis.), glutathione-S-transferase gene (Pharmacia, Piscataway, N.J.), β-galactosidase gene, chloramphenicol acetyltransferase gene and E. coli Trp E gene (Ausubel et al., 1989, Current Protocols in Molecular Biology, Chapter 16, Green Publishing Associates and Wiley Interscience, NY).

Eucaryotic mRNA does not contain a Shine-Dalgarno sequence. Instead, the selection of the translational start codon is usually determined by its proximity to the cap at the 5′ end of an mRNA. The nucleotides immediately surrounding the start codon in eucaryotic mRNA influence the efficiency of translation. Accordingly, one skilled in the art can determine what nucleic acid sequences will increase translation of a leader protein encoded by the expression cassette of the invention. Such nucleic acid sequences are within the scope of the invention.

D) Vectors

Vectors that may be used include, but are not limited to, those able to be replicated in prokaryotes and eukaryotes. Vectors include, for example, plasmids, phagemids, bacteriophages, viruses, cosmids, and F-factors. The invention includes any vector into which the expression cassette of the invention may be inserted and replicated in vitro or in vivo. Specific vectors may be used for specific cells types. Additionally, shuttle vectors may be used for cloning and replication in more than one cell type. Such shuttle vectors are known in the art. The nucleic acid constructs may be carried extrachromosomally within a host cell or may be integrated into a host cell chromosome. Numerous examples of vectors are known in the art and are commercially available. (Sambrook and Russell, Molecular Cloning: A Laboratory Manual, 3rd edition (Jan. 15, 2001) Cold Spring Harbor Laboratory Press, ISBN: 0879695765; New England Biolabs, Beverly, Mass.; Stratagene, La Jolla, Calif.; Promega, Madison, Wis.; ATCC, Rockville, Md.; CLONTECH, Palo Alto, Calif.; Invitrogen, Carlsbad, Calif.; Origene, Rockville, Md.; Sigma, St. Louis, Mo.; Pharmacia, Peapack, N.J.; USB, Cleveland, Ohio). These vectors also provide many promoters and other regulatory elements that those of skill in the art may include within the nucleic acid constructs of the invention through use of known recombinant techniques.

Examples of Vectors Useful in Bacteria

A nucleic acid construct for use in a prokaryote host, such as bacteria, will preferably include a replication system allowing it to be maintained in the host for expression or for cloning and amplification. In addition, a nucleic acid construct may be present in the cell in either high or low copy number. Generally, about 5 to about 200, and usually about 10 to about 150 copies of a high copy number nucleic acid construct will be present within a host cell. A host containing a high copy number plasmid will preferably contain at least about 10, and more preferably at least about 20 plasmids. Generally, about 1 to 10, and usually about 1 to 4 copies of a low copy number nucleic acid construct will be present in a host cell. The copy number of a nucleic acid construct may be controlled by selection of different origins of replication according to methods known in the art. Sambrook and Russell, Molecular Cloning: A Laboratory Manual, 3rd edition (Jan. 15, 2001) Cold Spring Harbor Laboratory Press, ISBN: 0879695765.

A nucleic acid construct containing an expression cassette can be integrated into the genome of a bacterial host cell through use of an integrating vector. Integrating vectors usually contain at least one sequence that is homologous to the bacterial chromosome that allows the vector to integrate. Integrations are thought to result from recombinations between homologous DNA in the vector and the bacterial chromosome. For example, integrating vectors constructed with DNA from various Bacillus strains integrate into the Bacillus chromosome (EPO Publ. No. 127 328). Integrating vectors may also contain bacteriophage or transposon sequences.

Extrachromosomal and integrating nucleic acid constructs may contain selectable markers to allow for the selection of bacterial strains that have been transformed. Selectable markers can be expressed in the bacterial host and may include genes that render bacteria resistant to drugs such as ampicillin, chloramphenicol, erythromycin, kanamycin (neomycin), and tetracycline (Davies et al., Ann. Rev. Microbiol. 32: 469, 1978). Selectable markers may also include biosynthetic genes, such as those in the histidine, tryptophan, and leucine biosynthetic pathways.

Numerous vectors, either extra-chromosomal or integrating vectors, have been developed for transformation into many bacteria. For example, vectors have been developed for the following bacteria: B. subtilis (Palva et al., Proc. Natl. Acad. Sci. USA, 79: 5582 (1982); EPO Publ. Nos. 036 259 and 063 953; PCT Publ. No. WO 84/04541), E. coli (Shimatake et al., Nature, 292: 128 (1981); Amann et al., Gene, 40: 183 (1985); Studier et al., J. Mol. Biol., 189: 113 (1986); EPO Publ. Nos. 036 776, 136 829 and 136 907), Streptococcus cremoris (Powell et al., Appl. Environ. Microbiol. 54: 655 (1988)); Streptococcus lividaits (Powell et al., Appl. Environ. Microbiol., 54: 655, (1988)), and Streptonyces lividans (U.S. Pat. No. 4,745,056). Numerous vectors are also commercially available (New England Biolabs, Beverly, Mass.; Stratagene, La Jolla, Calif.).

Examples of Vectors Useful in Yeast

Many vectors may be used to construct a nucleic acid construct that contains an expression cassette of the invention and that provides for the expression of a leader protein in yeast. Such vectors include, but are not limited to, plasmids and yeast artificial chromosomes. Preferably the, vector has two replication systems, thus allowing it to be maintained, for example, in yeast for expression and in a prokaryotic host for cloning and amplification. Examples of such yeast-bacteria shuttle vectors include YEp24 (Botstein, et al.; Gene, 8:17 (1979)), pCl/1 (Brake et al., Proc. Natl. Acad. Sci. USA, 81:4642 (1984)), and YRp17 (Stinchcomb et al., J. Mol. Biol., 158:157 (1982)). A vector may be maintained within a host cell in either high or low copy number. For example, a high copy number plasmid will generally have a copy number ranging from about 5 to about 200, and usually about 10 to about 150. A host containing a high copy number plasmid will preferably have at least about 10, and more preferably at least about 20. Either a high or low copy number vector may be selected, depending upon the effect of the vector and the leader protein on the host. (Brake et al., Proc. Natl. Acad. Sci. USA, 81:4642 (1984)).

A nucleic acid construct may also be integrated into the yeast genome with an integrating vector. Integrating vectors usually contain at least one sequence homologous to a yeast chromosome that allows the vector to integrate, and preferably contain two homologous sequences flanking an expression cassette of the invention. Integrations appear to result from recombinations between homologous DNA in the vector and the yeast chromosome. (Orr-Weaver et al., Methods in Enzymol., 101:228 (1983)). An integrating vector may be directed to a specific locus in yeast by selecting the appropriate homologous sequence for inclusion in the vector. One or more nucleic acid constructs may integrate, which may affect the level of recombinant protein produced. (Rine et al., Proc. Natl. Acad. Sci. USA, 80:6750 (1983)). The chromosomal sequences included in the vector can occur either as a single segment in the vector, which results in the integration of the entire vector, or two segments homologous to adjacent segments in the chromosome and flanking an expression cassette included in the vector, which can result in the stable integration of only the expression cassette.

Extrachromosomal and integrating nucleic acid constructs may contain selectable markers that allow for selection of yeast strains that have been transformed. Selectable markers may include, but are not limited to, biosynthetic genes that can be expressed in the yeast host, such as ADE2, HIS4, LEU2, TRP1, and ALG7, and the G418 resistance gene, which confer resistance in yeast cells to tunicamycin and G418, respectively. In addition, a selectable marker may also provide yeast with the ability to grow in the presence of toxic compounds, such as metal. For example, the presence of CUP1 allows yeast to grow in the presence of copper ions. (Butt et al., Microbiol. Rev. 51:351 (1987)).

Many vectors have been developed for transformation into many yeasts. For example, vectors have been developed for the following yeasts: Candida albicans (Kurtz et al., Mol. Cell. Biol. 6:142 (1986)), Candida maltose (Kunze et al., J. Basic Microbiol. 25:141 (1985)), Hansenula polymorpha (Gleeson et al., J. Gen. Microbiol., 132:3459 (1986); Roggenkamp et al., Mol. Gen. Genet., 202:302 (1986), Kluyveromyces fragilis (Das et al., J. Bacteriol., 158: 1165 (1984)), Kluyveromyces lactis (De Louvencourt et al., J. Bacteriol., 154:737 (1983); van den Berg et al., Bio/Technology 8:135 (1990)), Pichia guillerimondii (Kunze et al., J. Basic Microbiol., 25:141,(1985)), Pichia pastoris (Cregg et al., Mol. Cell. Biol. 5: 3376 (1985); U.S. Pat. Nos. 4,837,148 and 4,929,555), Saccharomyces cerevisiae (Hinnen et al., Proc. Natl. Acad. Sci. USA, 75:1929 (1978); Ito et al., J. Bacteriol., 153:163 (1983)), Schizosaccizaromyces pomrbe (Beach and Nurse, Nature, 300:706 (1981)), and Yarrowia lipolytica (Davidow et al., Curr. Genet., 10:39 (1985); Gaillardin et al., Curr. Genet., 10:49 (1985)).

Examples of Vectors Useful in Insect Cells

Baculovirus vectors have been developed for infection into several insect cells and may be used to produce nucleic acid constructs that contain an expression cassette of the invention. For example, recombinant baculoviruses have been developed for Aedes aegypti, Autographa calfornica, Bombyx mori, Drosophila melanogaster, Spodoptera frugiperda, and Trichoplusia ni (PCT Pub. No. WO 89/046699; Carbonell et al., J. Virol., 56:153 (1985); Wright, Nature, 321: 718 (1986); Smith et al., Mol. Cell. Biol., 3: 2156 (1983); and see generally, Fraser et al., In Vitro Cell. Dev. Biol. 25:225 (1989)). Such a baculovirus vector may be used to introduce an expression cassette into an insect and provide for the expression of a leader protein within the insect cell.

Methods to form a nucleic acid construct having an expression cassette of the invention inserted into a baculovirus vector are well known in the art. Briefly, an expression cassette of the invention is inserted into a transfer vector, usually a bacterial plasmid which contains a fragment of the baculovirus genome, through use of common recombinant methods. The plasmid may also contain a polyhedrin polyadenylation signal (Miller et al., Ann Rev. Microbiol., 42:177 (1988) and a prokaryotic selection marker, such as ampicillin resistance, and an origin of replication for selection and propagation in Escherichia coli. A convenient transfer vector for introducing foreign genes into AcNPV is pAc373. Many other vectors, known to those of skill in the art, have been designed. Such a vector is pVL985 (Luckow and Summers, Virology 17:31 (1989)).

A wild-type baculoviral genome and the transfer vector having an expression cassette insert are transfected into an insect host cell where the vector and the wild-type viral genome recombine. Methods for introducing an expression cassette into a desired site in a baculovirus virus are known in the art (Summers and Smith, Texas Agricultural Experiment Station Bulletin No. 1555, 1987. Smith et al., Mol. Cell. Biol., 3:2156 (1983); and Luckow and Summers, Virology, 17:31 (1989)). For example, the insertion can be into a gene such as the polyhedrin gene, by homologous double crossover recombination; insertion can also be into a restriction enzyme site engineered into the desired baculovirus gene (Miller et al., Bioessays, 4:91 (1989)). The expression cassette, when cloned in place of the polyhedrin gene in the nucleic acid construct, will be flanked both 5′ and 3′ by polyhedrin-specific sequences. An advantage of inserting an expression cassette into the polyhedrin gene is that occlusion bodies resulting from expression of the wild-type polyhedrin gene may be eliminated. This may decrease contamination of leader proteins produced through expression and formation of occlusion bodies in insect cells by wild-type proteins that would otherwise form occlusion bodies in an insect cell having a functional copy of the polyhedrin gene.

The packaged recombinant virus is expressed and recombinant plaques are identified and purified. Materials and methods for baculovirus and insect cell expression systems are commercially available in kit form. (Invitrogen, San Diego, Calif., USA (“MaxBac” kit)). These techniques are generally known to those skilled in the art and fully described in Summers and Smith, Texas Agricultural Experiment Station Bulletin No.1555, 1987.

Plasmid-based expression systems have also been developed the may be used to introduce an expression cassette of the invention into an insect cell and produce a leader protein (McCarroll and King, Curr. Opin. Biotechnol., 8:590 (1997)). These plasmids offer an alternative to the production of a recombinant virus for the production of leader proteins.

Examples of Vectors Useful in Mammalian Cells

An expression cassette of the invention may be inserted into many mammalian vectors that are known in the art and are commercially available. (CLONTECH, Carlsbad, Calif.; Promega, Madision, Wis.; Invitrogen, Carlsbad, Calif.). Such vectors may contain additional elements such as enhancers and introns having functional splice donor and acceptor sites. Nucleic acid constructs may be maintained extrachromosomally or may integrate in the chromosomal DNA of a host cell. Mammalian vectors include those derived from animal viruses, which require trans-acting factors to replicate. For example, vectors containing the replication systems of papovaviruses, such as SV40 (Gluzman, Cell, 23:175 (1981)) or polyomaviruses, replicate to extremely high copy number in the presence of the appropriate viral T antigen. Additional examples of mammalian vectors include those derived from bovine papillomavirus and Epstein-Barr virus. Additionally, the vector may have two replication systems, thus allowing it to be maintained, for example, in mammalian cells for expression and ill a prokaryotic host for cloning and amplification. Examples of such mammalian-bacteria shuttle vectors include pMT2 (Kaufman et al., Mol. Cell. Biol. 9:946 (1989)) and pHEBO (Shimizu et al., Mol. Cell. Biol. 6:1074-(1986)).

E) Host Cells

Host cells producing the recombinant precursor polypeptides for the methods of the invention include prokaryotic and eukaryotic cells of single and multiple cell organisms. Bacteria, fungi, plant, insect, vertebrate and its subclass mammalian cells and organisms may be employed. Single cell cultures from such sources as well as functional tissue and whole organisms can operate as production hosts according to the invention. Examples include E. coli, tobacco plant culture, maize, soybean, fly larva, mice, rats, hamsters, as well as CHO cell cultures, immortal cell lines and the like.

In a preferred embodiment, bacteria are used as host cells. Examples of bacteria include, but are not limited to, Gram-negative and Gram-positive organisms. Escherichia coli is a preferred organism for expression of preselected polypeptides and amplification of nucleic acid constructs. Many publicly available E. coli strains include K-strains such as MM294 (ATCC 31, 466); X1776 (ATCC 31, 537); KS 772 (ATCC 53, 635); JM109; MC1061; HMS174; and the B-strain BL21. Recombination minus strains may be used for nucleic acid construct amplification to avoid recombination events. Such recombination events may remove concatemers of open reading frames as well as cause inactivation of an expression cassette. Furthermore, bacterial strains that do not express a select protease may also be useful for expression of preselected polypeptides to reduce proteolytic processing of expressed polypeptides. Such strains include, for example, Y1090hsdR, which is deficient in the ion protease.

Eukaryotic cells may also be used to produce a preselected polypeptide and for amplifying a nucleic acid construct. Eukaryotic cells are useful for producing a preselected polypeptide when additional cellular processing is desired. For example, a preselected polypeptide may be expressed in a eukaryotic cell when glycosylation of the polypeptide is desired. Examples of eukaryotic cell lines that may be used include, but are,not limited to: AS52, H187, mouse L cells, NIH-3T3, HeLa, Jurkat, CHO-K1, COS-7, BHK-21, A-431, HEK293, L6, CV-1, HepG2, HC11, MDCK, silkworm cells, mosquito cells, and yeast.

F) Transformation

Methods for introducing exogenous DNA into bacteria are available in the art, and usually include either the transformation of bacteria treated with CaCl₂ or other agents, such as divalent cations and DMSO. DNA can also be introduced into bacterial cells by electroporation, use of a bacteriophage, or ballistic transformation. Transformation procedures usually vary with the bacterial species to be transformed (see, e.g., Masson et al., FEMS Microbiol. Lett. 60: 273 (1989); Palva et al., Proc. Natl. Acad. Sci. USA, 79: 5582 (1982); EPO Publ. Nos. 036 259 and 063 953; PCT Publ. No. WO 84/04541 [Bacillus], Miller et al., Proc. Natl. Acad. Sci. USA, 8: 856 (1988); Wang et al., J. Bacteriol., 172: 949 (1990) [Campylobacter], Cohen et al., Proc. Natl. Acad. Sci. USA, 69: 2110 (1973); Dower et al., Nuc. Acids Res., 16: 6127 (1988); Kushner, “An improved method for transformation of Escherichia coli with ColE1-derived plasmids”, in: Genetic Engineering: Proceedings of the International Symposium on Genetic Engineering (eds. H. W. Boyer and S. Nicosia), 1978; Mandel et al., J. Mol. Biol. 53: 159 (1970); Taketo, Biochim. Biophys. Acta, 949: 318 (1988) [Escherichia], Chassy et al., FEMS Microbiol. Lett., 44: 173, (1987) [Lactobacillus], Fiedler et al., Anal. Biochen, 170: 38 (1988) [Pseudomonas], Augustin et al., FEMS Microbiol. Lett., 66: 203 (1990) [Staphylococcus), Barany et al., J. Bacteriol., 144: 698 (1980); Harlander, “Transformation of Streptococcus lactis by electroporation”, in: Streptococcal Genetics (ed. J. Ferretti and R. Curtiss III), 1987; Perry et al., Infec. Immun., 32: 1295 (1981); Powell et al., Appl. Environ. Microbiol., 54: 655 (1988); Somkuti et al., Proc. 4th Eur. Cong. Biotechnology, 1: 412 (1987) [Streptococcus]).

Methods for introducing exogenous DNA into yeast hosts are well-known in the art, and usually include either the transformation of spheroplasts or of intact yeast cells treated with alkali cations. Transformation procedures usually vary with the yeast species to be transformed (see, e.g., Kurtz et al., Mol. Cell. Biol., 6:142 (1986); Kunze et al., J. Basic Microbiol., 25:141 (1985) [Candida], Gleeson et al., J. Gen. Microbiol., 132:3459 (1986); Roggenkamp et al., Mol. Gen. Genet., 202:302 (1986) [Hansenula], Das et al., J. Bacteriol., 158:1165 (1984); De Louvencourt et al., J. Bacteriol., 754:737 (1983); Van den Berg et al., Bio/Technology, 8:135 (1990) [Kluyveromyces], Cregg et al., Mol. Cell. Biol., 5:3376 (1985); Kunze et al., J. Basic Microbiol., 25:141 (1985); U.S. Pat. Nos. 4,837,148 and 4,929,555 [Pichia], Hinnen et al., Proc. Natl. Acad. Sci. USA, 75:1929 (1978); Ito et al., J. Bacteriol., 153:163 (1983) [Saccharomyces], Beach and Nurse, Nature 300:706 (1981) [Schizosaccharonzyces], and Davidow et al., Curr. Genet., 10:39 (1985); Gaillardin et al., Curr. Genet., 10:49 (1985) [Yarrowia]).

Exogenous DNA is conveniently introduced into insect cells through use of recombinant viruses, such as the baculoviruses described herein.

Methods for introduction of heterologous polynucleotides into mammalian cells are known in the art and include lipid-mediated transfection, dextran-mediated transfection, calcium phosphate precipitation, polybrene-mediated transfection, protoplast leader, electroporation, encapsulation of the polynucleotide(s) in liposomes, biollistics, and direct microinjection of the DNA into nuclei. The choice of method depends on the cell being transformed as certain transformation methods are more efficient with one type of cell than another. (Feigner et al., Proc. Natl. Acad. Sci., 84:7413 (1987); Feigner et al., J. Biol. Chem., 269:2550 (1994); Graham and van der Eb, Virology 52:456 (1973); Vaheri and Pagano, Virology, 27:434 (1965); Neuman et al., EMBO J., 1:841 (1982); Zimmerman, Biochem. Biophys. Acta, 694:227 (1982); Sanford et al., Methods Enzymol., 217:483 (1993); Kawai and Nishizawa, Mol. Cell. Biol. 4:1172 (1984); Chaney et al., Somat. Cell Mol. Genet., 12:237 (1986); Aubin et al., Methods Mol. Biol., 62:319 (1997)). In addition, many commercial kits and reagents for transfection of eukaryotic are available.

Following transformation or transfection of a nucleic acid into a cell, the cell may be selected for through use of a selectable marker. A selectable marker is generally encoded on the nucleic acid being introduced into the recipient cell. However, co-transfection of selectable marker can also be used during introduction of nucleic acid into a host cell. Selectable markers that can be expressed in the recipient host cell may include, but are not limited to, genes which render the recipient host cell resistant to drugs such as actinomycin C₁, actinomycin D, amphotericin, ampicillin, bleomycin, carbenicillin,. chloramphenicol, geneticin, gentamycin, hygromycin B, kanamycin monosulfate, methotrexate, mitomycin C, neomycin B sulfate, novobiocin sodium salt, penicillin G sodium salt, puromycin dihydrochloride, rifampicin, streptomycin sulfate, tetracycline hydrochloride, and erythromycin. (Davies et al., Ann. Rev. Microbiol., 32: 469, 1978). Selectable markers may also include biosynthetic genes, such as those in the histidine, tryptophan, and leucine biosynthetic pathways. Upon transfection or transformation of a host cell, the cell is placed into contact with an appropriate selection marker.

For example, if a bacterium is transformed with a nucleic acid construct that encodes resistance to ampicillin, the transformed bacterium maybe placed on an agar plate containing ampicillin. Thereafter, cells into which the nucleic acid construct was not introduced would be prohibited from growing to produce a colony while colonies would be formed by those bacteria that were successfully transformed.

### EXAMPLES

The following series of Examples illustrates procedures for cloning, expression and detection of a precursor polypeptide that can be used to generate a peptide of interest. Examples 1 though 5 provide the protocol and experimental procedures used for preparing a peptide of interest using the clostripain cleavage techniques of the present invention. Example 6 provides the application of these protocols and procedures to a specific peptide. The peptide chosen is GLP-1(7-36)NH₂. Example 7 provides data showing the parameters for affecting selectivity of the clostripain cleavage. This series of examples are intended to illustrate certain aspects of the production of GLP-1 and analogs thereof and are not intended to be limiting thereof.

### Example 1

### Construction of a Vector that Contains DNA Which Encodes a Desired Precursor Polypeptide

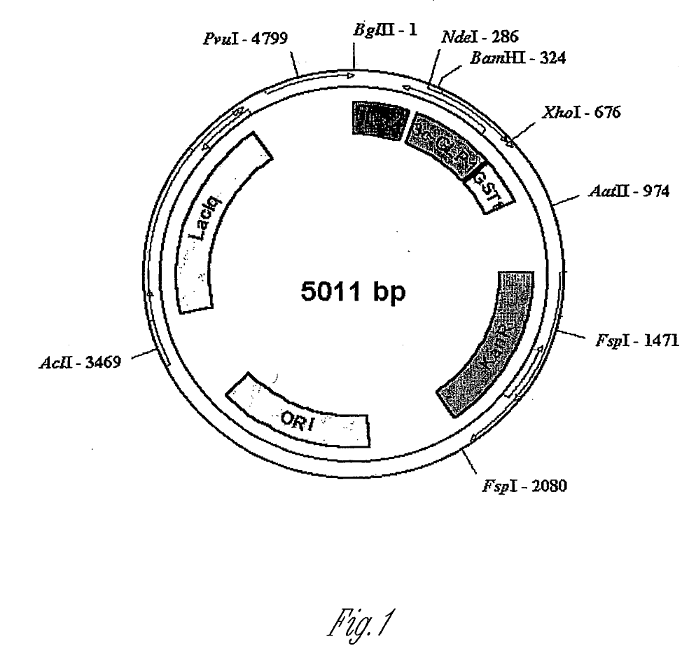

In order to express the desired precursor polypeptide, a preferred expression vector, pBN122, was constructed through use of PCR, restriction enzyme digestion, DNA ligation, transformation into a bacterial host, and screening procedures according to procedures described, for example, in Sambrook et al., Molecular Cloning (2^{nd} edition). Preferably the vector contains regulatory elements that provide for high level expression of a desired precursor polypeptide. Examples of such regulatory elements include, but are not limited to: an inducible promoter such as the chlorella virus promoter (U.S. Pat. No. 6,316,224); an origin of replication for maintaining the vector in high copy number such as a modified pMB1 promoter, a LaqIq gene for promoter suppression; an aminophosphotransferase gene for kanamycin resistance; and a GST terminator for terminating mRNA synthesis. (FIG. 1).

E. coli is a preferred host. To clone the expression cassette for the production of T7-tag-CS-[GPGDR-GLP-1(7-36)-AFL]₃ (SEQ ID NO:9), PCR or multiple PCR extension was performed to synthesize a DNA sequence encoding the T7tag, and GLP-1(7-36) gene using preferred codons for E. coli. DNA providing the T7 gene 10 ribosome binding site and the first twelve amino acids (T7tag) after the initiation codon was cloned into plasmid pBN122 at XbaI-SalI sites between the promoter and the terminator. DNA encoding GLP-1(7-36) was cloned into the above plasmid at SalI-XhoI sites. Plasmids were transformed into E. coli using heat shock or electroporation procedures (2^{nd} edition, Sambrook et. al). Cells were streaked onto LB+Kanamycin+agar plates, cultures were grown in LB+Kanamycin media from single colonies. Plasmids from these cultures were prepared, screened by restriction enzyme digestion, and sequenced using DNA sequencers. The cultures with the correct plasmid sequence were saved in glycerol stock at −80° C. or below.

Alternative peptides can be cloned by this method using different combinations of restriction enzymes and restriction sites according to methods known in the art.

### Example 2

### Expression of the Precursor Polypeptide

A shaking flask was inoculated from a glycerol stock of an E. coli strain containing a pBN121 plasmid encoding the desired polypeptide. A complex media containing 1% tryptone was employed that was supplemented with glucose and kanamycin. The shaking culture was grown in a rotary shaker at 37° C. until the optical density was 1.5±0.5 at 540 nm. The contents of the shaking flask culture were then used to inoculate a 5 L fermentation tank containing a defined minimal: media containing magnesium, calcium, phosphate and an assortment of trace metals. Glucose served as the carbon source. Kanamycin was added to maintain selection of the recombinant plasmid. During fermentation, dissolved oxygen was controlled at 40% by cascading agitation and areation with additional oxygen. A solution of ammonium hydroxide was used to control the pH at about pH 6.9.

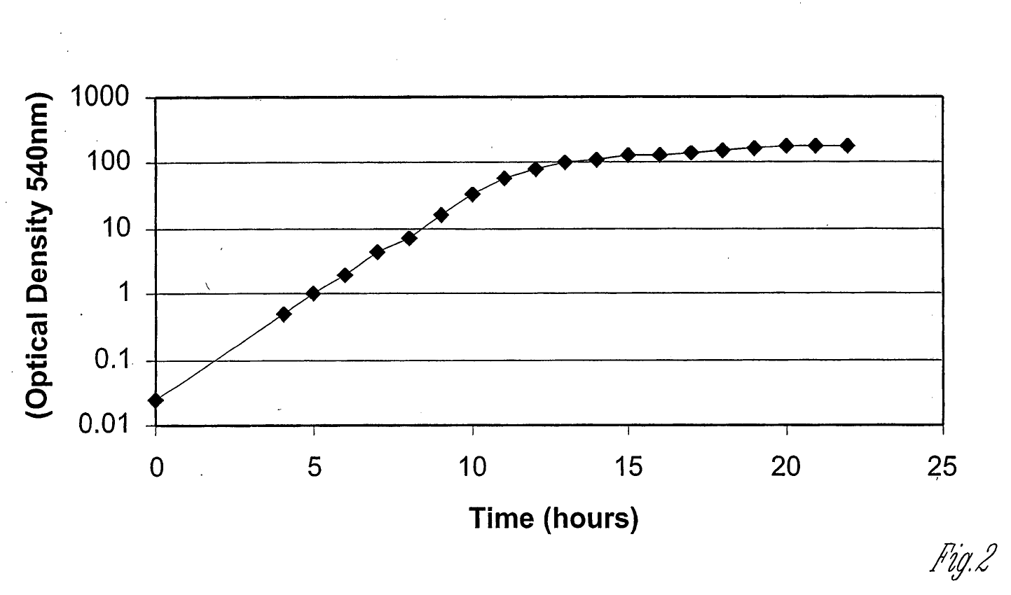

Cell growth was monitored at 540 nm until a target optical density of between about 75 OD, was reached and isopropyl-β-D-thiogalatoside (IPTG at between 0.1 and 1.0 mM) was added to induce expression of the desired polypeptide. (FIG. 2). When induction was complete, the cells were cooled in the fermenter and harvested with a continuous flow solid bowl centrifuge. The sedimented cells were frozen until used.

The frozen cell pellet was thawed and homogenized in 50 mM Tris, 2.5 mM EDTA, pH 7.8. Inclusion bodies were washed in water and were collected by solid bowl centrifuigation. Alternatively, cells were suspended in 8 M urea then lysed by conventional means and then centrifuged. The supernatant fluid contained the precursor peptide.

### Example 3

### Detection of Precursor Polypeptides

To monitor the production of the precursor polypeptide preparation, 100 μL of sample (fermentation culture or from a purification process step) was dissolved in 1 mL 71% phenol, 0.6 M citric acid, vortexed and bath sonicated briefly. The dissolved sample was diluted 12.5-fold to 50-fold in 50% acetonitrile, 0.09% TFA, and centrifuged to render it compatible with the chromatography system to be employed. The dissolved precursor polypeptide and E. coli cell products remain soluble in the diluted solution, while other insoluble matters are removed.

The samples were then analyzed using a tapered, 5 micron Magic Bullet C4 column (Michrom BioResources). The absolute peak area of the precursor polypeptide was obtained by recording the absorbance at 280 nm as a function of time. The TPLC method was as follows:
1. Mobile phase: A—0.1% TFA in water, B—0.08% TFA in acetonitrile.
2. Detection: 280 nm.
3. Gradient: 1 mL/min. at 50° C., using 10-90% B(2.5 min.), 90-10% B(0.1 min.), 10% B(1.4 min.). The gradient may be modified for better separation of different precursor peptides.
4. Injection: 1-10 μL.

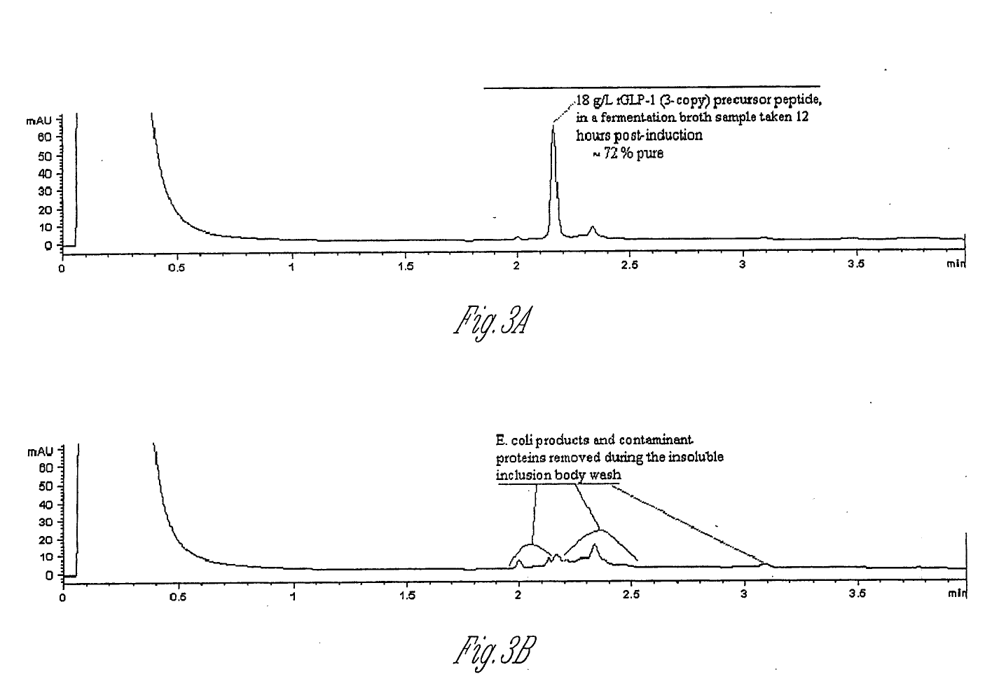

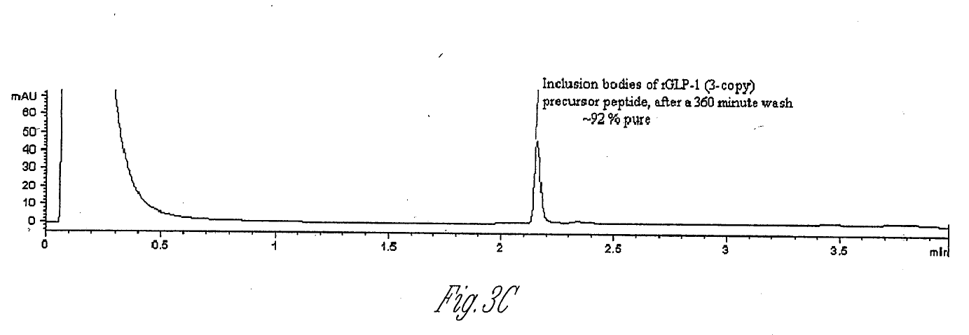

The precursor polypeptide peak area is compared to the peak area from a reference polypeptide standard chromatographed under the same conditions. The precursor polypeptide concentration is determined by normalizing for the different calculated molar absorptivities (ε_{280 nm}) of a standard and the precursor polypeptide, injection volumes, and dilution factors. The results of these analyses are presented in FIG. 3.

### Example 4

### Preparation and Cleavage of Precursor Polypeptides

Approximately 100 grams of E. coli cells containing the desired precursor polypeptide were lysed by combining them with approximately two liters of 8 M urea containing 0.1 M NH₄OH, pH 10.0 (adjusted with reagent grade HCl). This treatment caused the cells to lyse and produce a cell free extract. Alternatively, cells can be lysed with 8 M urea at neutral or acidic pH. Lysis methods utilizing urea are preferably used to lyse cells that express soluble precursor polypeptides.

In one example, the lysate was homogenized for 3 minutes using a commercial homogenizer. The suspension was then centrifuged for 45 minutes at 16,900×g. The supernatant fluid was diluted to a final protein concentration of from 0.1 to 2 mg/ml in 50 mM HEPES buffer, containing 1 mM CaCl₂ and 1 mM cysteine. Alternately the lysate was subjected to tangential flow filtration (TFF) using an 8 kD exclusion membrane. The loss in the filtered volume was replaced with 50 mM HEPES containing 0-3 M urea, 1 mM CaCl₂, and 1 mM cysteine, pH 6.0-6.9.

For cells containing-the plasmid that expresses T7-tag-GS-[GPGDR-GLP-1(7-36)-AFL]₃ (SEQ ID NO:9) in inclusion bodies, cell lysis was preferably performed by sonication or mechanical in 50 mM Tris, 2.5 mM EDTA, pH 7.5. Centrifuigation was then performed to sediment the inclusion bodies. After the supernatant fluid was decanted, the pellet was resuspended and pelleted three times in distilled water to wash the inclusion bodies. The pelleted inclusion bodies were then dissolved in 6 M urea, mechanically homogenized for 2 min and then centrifuged to remove the insoluble material. The pellet was then resuspended in a buffer containing 1.8 M NH₄OH, about 2 M urea, 1 mM CaCl₂, 1 mM cysteine, about 1.4 mg/ml GLP-1, and about 20 units of clostripain per mg of precursor polypeptide (pH about 9.3, adjusted with HCl). The mixture was incubated at 45° C. for a period of about 120 minutes and then the cleavage reaction was terminated by the addition of EDTA to a final concentration of about 10 mM. Alternatively the reaction was terminated when the concentration of the precursor polypeptide was less than 10% of the starting concentration. This procedure has been conducted with 4 kilograms of inclusion bodies to produce about 100 grams of highly purified GLP-1(7-36)NH₂.

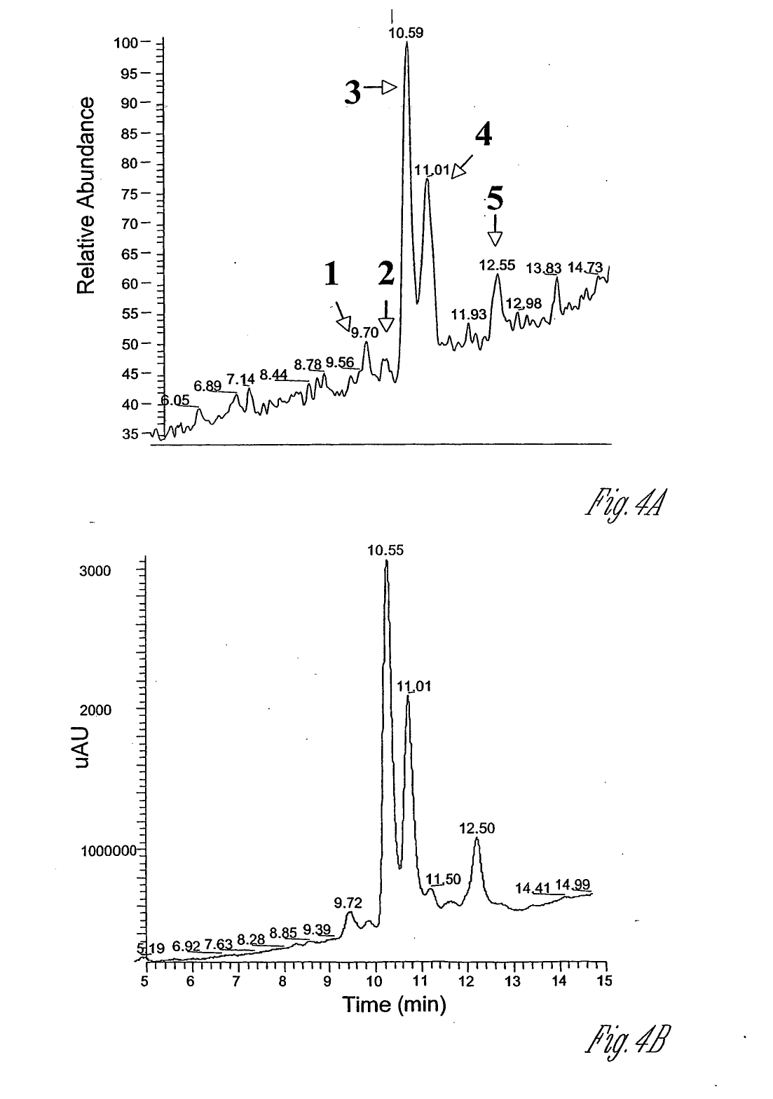

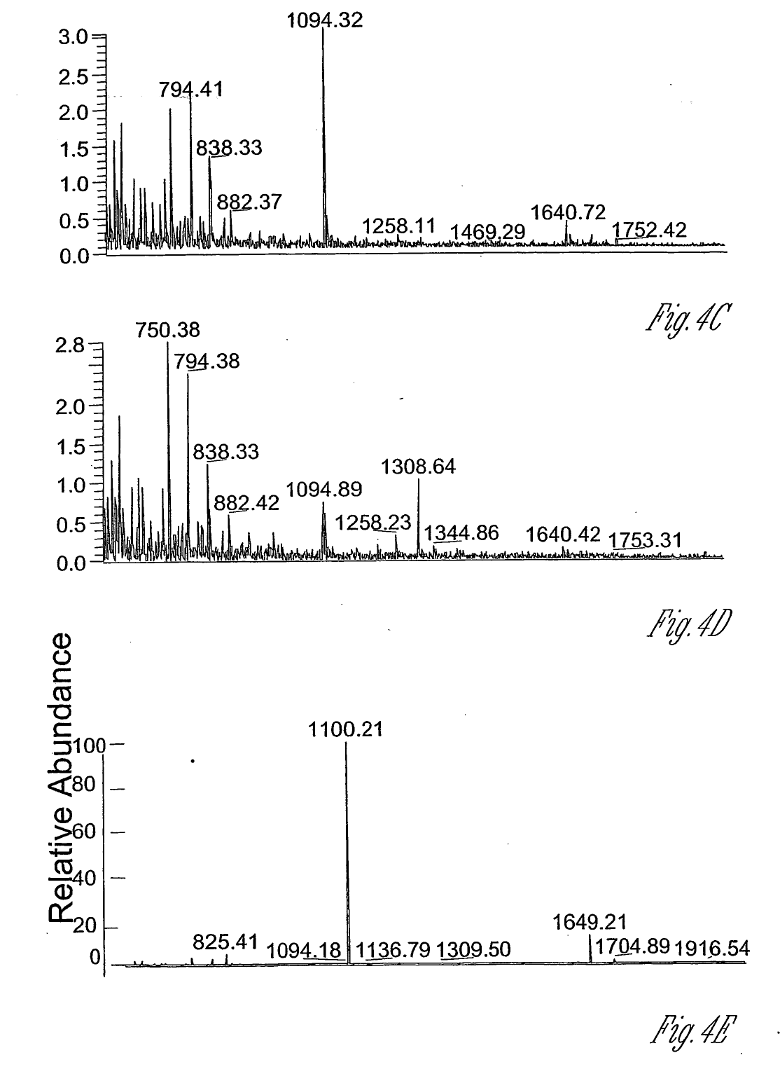

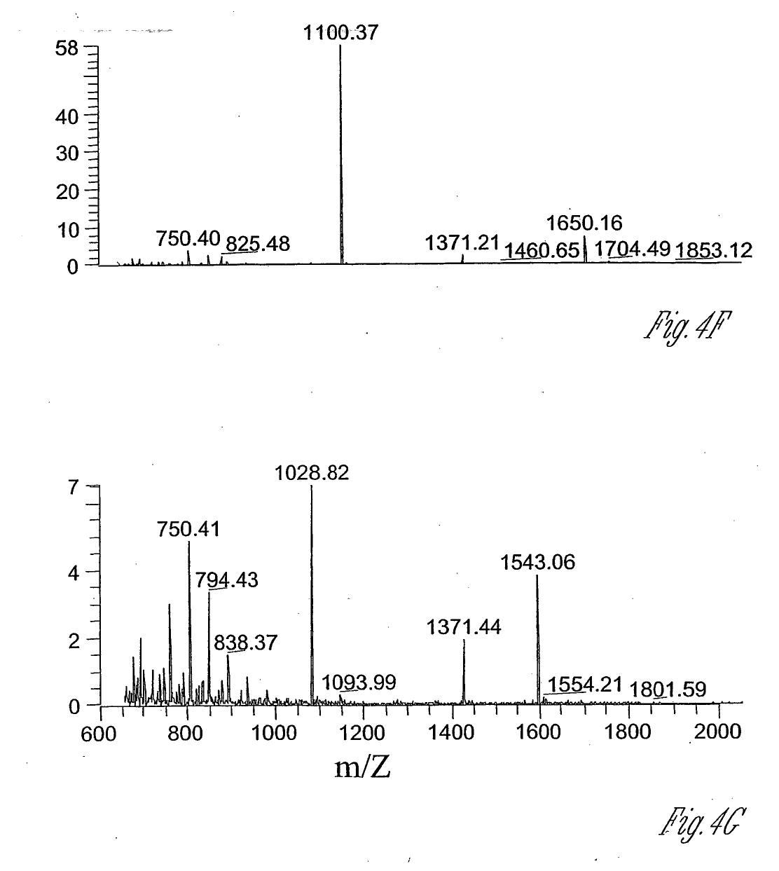

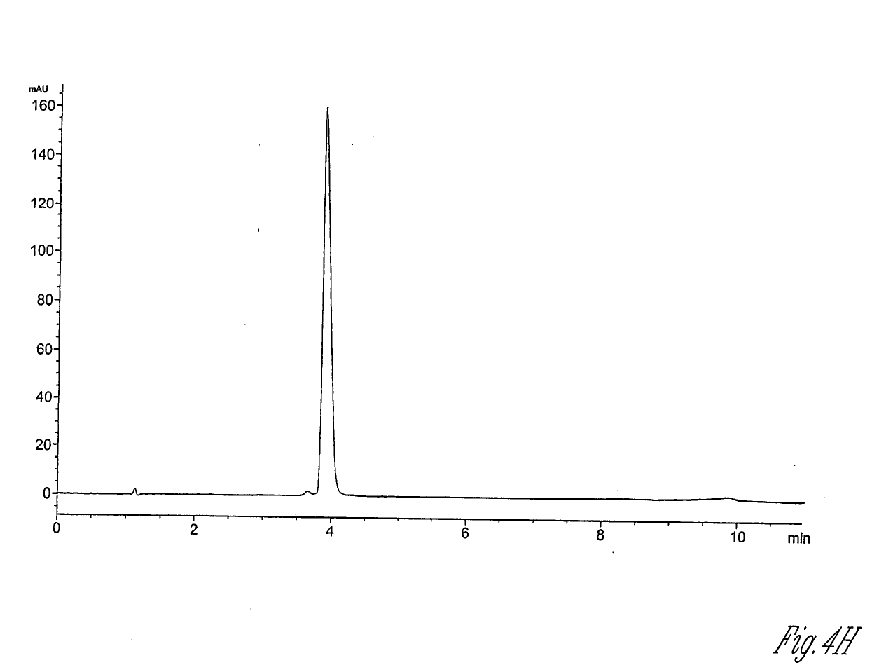

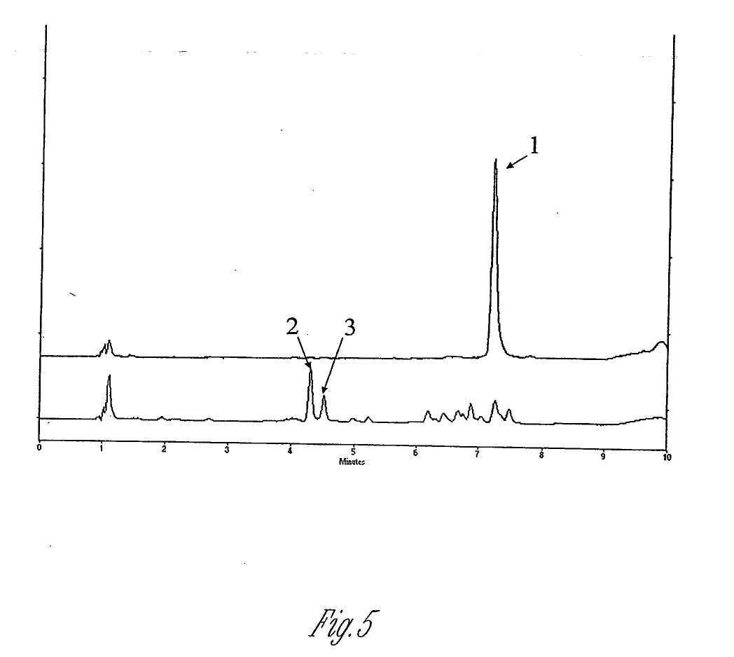

Throughout the course of the reaction, 30 μl aliquots were withdrawn and quenched by the addition of EDTA to a final concentration of 10 mM. 5 ul samples of each quenched aliquot were then injected into a Finnegan LCQ DUO ion trap mass spectrometer equipped with a Waters Symmetry C18 column operating in a positive ion electrospray mode for analysis. During the sampling period, molecular weight determination was performed by full scan mass spectrometry. Typical MS conditions included a scan range of 300-2000 Da/e. The results of these analyses are presented in FIG. 4.

LC analysis was performed on a system consisting of a Xcaliber software, ThermoQuest Surveyor MS pumps, a ThermoQuest Surveyor UV spectrophotometric PDA detector and a ThermoQuest Surveyor autosampler. The parameters of the chromatographic column are indicated below.

| | |
|---|---|
| Column: Manufacturer: | Waters Company |
| Packing support: | Symmetry C18 |
| Particle size: | 3.5 μm |
| Pore size: | 100 Å |
| Column size: | 2.1 × 150 mm |
| Guard column: | 3.5 μm, 2.1 × 10 mm |

Chromatographic conditions were: flow-rate 300 uL/min and buffers A: 0.1% TFA, B: acetonitrile, 0.08% TFA. The gradient was from 15% B to 30% B in 3 min, to 55% B in 19 min, to 90% B in 3 min, temperature 50° C. Detection was over the range 210-320 nm on the PDA detector, Channel A 214 nm, channel B 280 nm. Mass detection was over the 300-2000 Da/e range. All the samples were analyzed on an LCQ-DUO ESI mass spectrometer. Usually, the masses observed with significant relative abundance are the doubly or triply charged ions, i.e. [M+2H ²⁺/2 or [M+3H ]³⁺/3. The complete mass spectrum as a function of time could be evaluated following the chromatographic procedure through use of the system software. This allows for analysis of individual peaks that eluted from the column.

The results shown in FIG. 4A illustrate that the identity of peptides produced in a cleavage reaction can be identified. FIG. 4H illustrates the production of GLP-1 from 2 kilograms of inclusion bodies digested as described above and subsequently purified by column chromatography. The purity of the sample following chromatography was typically in excess of 90% and often greater than 98%. (FIG. 4H). The purified product also had the correct mass and amino acid composition and sequence.

### Example 5

### Preparation of Amidated Cleavage Products from a Multicopy Precursor Polypeptide

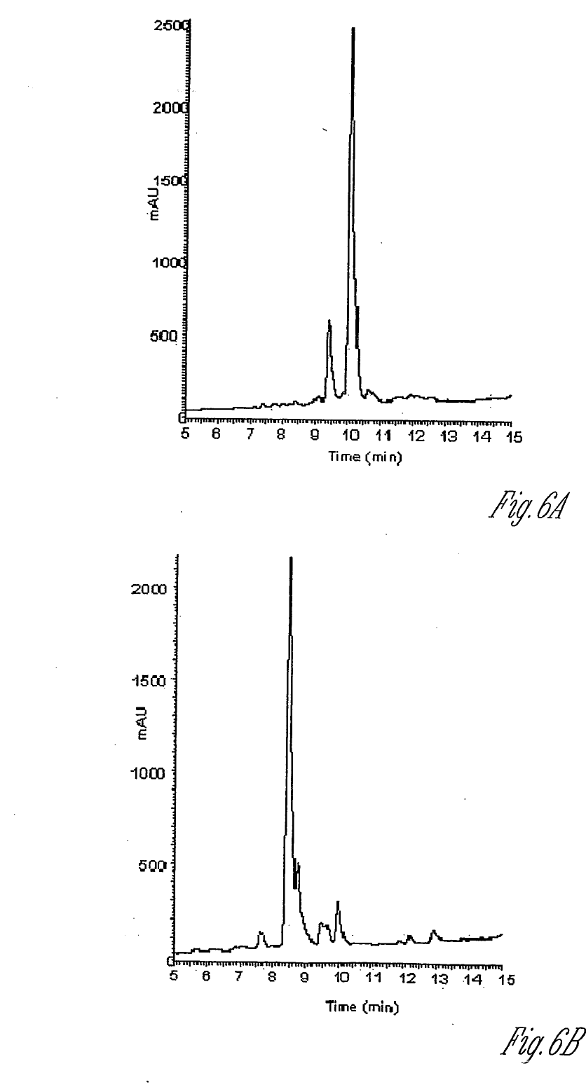

A reaction mixture was prepared by combining clostripain with T7-tag-GS-[GPGDR-GLP-1(7-36)-AFL]₃ (SEQ ID NO:9) (6.66 mg/ml) in a cleavage reaction containing 2.8 M NH₄Cl, 1.0 M NH₄OH buffer with 1 mM CaCl₂ and 1 mM cysteine at pH 8.5-9.0 and 45° C. The cleavage reaction was initiated by the addition of clostripain (12 units per mg of precursor polypeptide) to the cleavage reaction. The reaction was quenched by diluting the cleavage reaction 10-fold in 60% acetic acid. The products of the reaction were analyzed with an HPLC that was equipped with a Vydac C4 column (FIG. 5). The following gradient was used: 30% buffer B for 7.5 minutes and 50-70% buffer B in 1 minute at a flow rate of 2.0 mL/min. The buffers were as follows: A: 5% acetonitrile and 0.1% TFA; B: 95% acetonitrile and 0.1% TFA. The injection volume was 20 μl for each sample. The products of the cleavage reaction were arnidated on the C-terminus and were produced with a 15% yield at pH 8.6 to 8.8. It is noted that NH₄Cl may be substituted for ammonia in order to cause C-terminal amidation of the cleavage products. A purified preparation of GLP-1(7-36)-NH₂ was subjected to complete amino acid sequence analysis which confirmed the structure of this peptide.

### Example 6

### Production of a Peptide Produced Through Transpeptidation

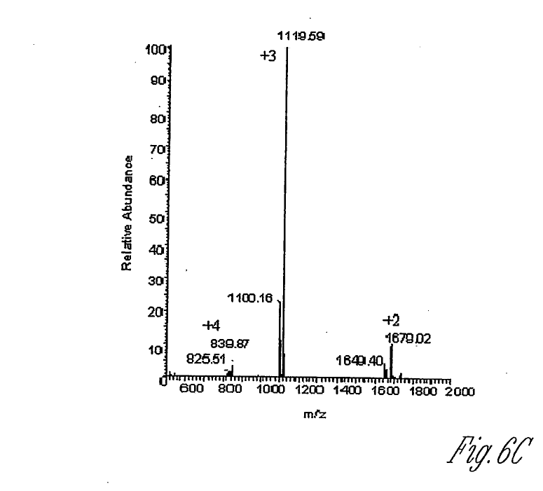

GLP-1(7-36)AFAHSe (SEQ ID NO:10) was expressed from a recombinant construct T7-Tag-M-[GLP-1(7-36)AFAM]₇ GLP-1(7-36)AFAMHAE (SEQ ID NO:11) and was then prepared by CNBr cleavage of the expression product from the expression construct. To perform the clostripain catalyzed cleavage and transpeptidation reaction, the following were combined in a 1 ml reaction mixture: 1 mg of the GLP-1(7-36)AFAHSe (SEQ ID NO:10; free acid and lactone mixture), 0.2 mM CaCl₂, 1 mM cysteine, 0.5 M glycine, 1.25 M NH₄OH and 1 unit of clostripain. The mixture was incubated for 30 minutes at pH 10.0 and 45° C. The reaction was terminated by diluting an aliquot of the reaction mixture 10-fold into 60% acetic acid. The sample was then subjected to analysis by LC/MS as described above. The data in FIG. 6A shows HPLC analysis of the GLP-1(7-36)AFAHSe (SEQ ID NO:10). The constituents at about 9.4 minutes and about 10.09 minutes were GLP-1(7-36)AFAHSe (SEQ ID NO:10) and GLP-1(7-36)AFAHSe-lactone (SEQ ID NO:12) respectively. After 30 minutes of incubation, the single major component that eluted at about 8.3 minutes (FIG. 6B) was detected with the concomitant disappearance of the two constituents of the unreacted material. The mass of the main constituent of FIG. 6B was 3356 (FIG. 6C) which was identical to the molecular weight of GLP-1(7-37). The yield was in excess of 60%. The method described may also be conducted in the absence of NH₄OH at a pH of between about 8.8 and 9.5. The transpeptidation methods described above can also be used to add amino acids other than glycine to the C-terminus of a peptide product produced through cleavage of a precursor polypeptide. Additionally, clostripain can be used to add other nucleophiles, such as alcohols and dipeptides, to the C-terminus of a cleaved peptide to form esters and extended peptides respectively. Also, clostripain can be used to add an amino acid analog to the C-terminus of a peptide product. The identity of the moiety added to the C-terminus is determined by the specificity of the clostripain binding site.

Wild type clostripain (different from recombinant clostripain) was purchased from a vendor (200 u/mg dry weight, Worthington). The dried enzyme was kept at 4° C. A stock solution was made by resolubilization of the dried enzyme in 25 mM HEPES buffer at pH 7.1 with 10 mM DTT and 5 mM CaCl₂ and was stored at 4° C. or in an ice bucket before use. Wild-type and recombinant clostripain produced equivalent results.

### Example 7

### Production of Variant Forms of GLP-1(7-36)NH₂ and GLP-1(7-37)

The methods described in Examples 1-6 can be used to produce nearly any variant of a GLP-1(7-36)NH₂ peptide. Examples of such variants include, but are not limited to, GLP-1(7-36)A2G, GLP-1(7-36)K26R and combinations thereof. For example, an expression construct can be constructed that expresses the T7-tag-GS-[GPGDR-GLP-1(7-36)K26R-AFL]₃ (SEQ ID NO:13) precursor polypeptide according to the method described in Example 1. This precursor polypeptide can be expressed and detected according to the methods described in Examples 2 and 3 and then cleaved according to the method of Example 4. The identification of GLP-1(7-36)K26R as the cleavage product can be conducted according to the methods described in Example 5. Accordingly, analogous methods can be used to create peptide products having virtually any desired amino acid substitution.

In addition, the transamidation methods described herein may be used to add glycine or amino acids other than glycine to the C-terminus of a peptide product produced through cleavage of a precursor polypeptide. As stated above, clostripain can be used to add other nucleophiles, such as alcohols and dipeptides, to the C-terminus of a cleaved peptide to form esters and extended peptides respectively. Also, clostripain can be used to add an amino acid analog to the C-terminus of a peptide product. Therefore, the methods disclosed herein provide for the production of a GLP-1(7-36)NH₂ as well as numerous variants thereof.

### REFERENCES

Alberts et al., Molecular Biology of the Cell, 2nd ed., 1989
Amann et al., Gene, 25:167 (1983)
Amann et al., Gene, 40: 183, (1985)
Aubin et al., Methods Mol. Biol., 62:319 (1997)
Augustin et al., FEMS Microbiol. Lett., 66: 203 (1990)
Ausubel et al., Current Protocols in Molecular Biology, Green Publishing Associates and Wiley Interscience, NY (1989)
Barany et al., J. Bacteriol., 144: 698 (1980)
Beach and Nurse, Nature, 300:706 (1981)
Beaucage and Caruthers, Tetra. Letts., 22:1859 (1981)
Boshart et al., Cell, 41:521 (1985)
Botstein, et al., Gene, 8:17 (1979)
Brake et al., Proc. Natl. Acad. Sci. USA, 81:4642 (1984)
Butt et al., Microbiol. Rev., 51:351 (1987)
Carbonell et al., Gene, 73: 409 (1988)
Carbonell et al., J. Virol., 56:153 (1985)
Catsimpoolas and Wood, J. Biol. Chem., (1979)
Chaney et. al., Somat. Cell Mol. Genet., 12:237 (1986)
Chang et al., Nature, 198:1056 (1977)
Chassy et al., FEMS Microbiol. Lett., 44: 173 (1987)
Cohen et al., Proc. Natl. Acad. Sci. USA, 69:.2110 (1973)
Cohen et al., Proc. Natl. Acad. Sci. USA, 77:1078 (1980)
Cregg et al., Mol. Cell. Biol., 5: 3376, (1985)
Das et al., J. Bacteriol., 158: 1165 (1984)
Davidow et al., Curr. Genet. 10:39 (1985)
Davies et al., Ann. Rev. Microbiol., 32: 469, 1978
Dayhoff et al., Atlas of Protein Sequence and Structure, Natl. Biomed. Res. Found., Washington, C.D. (1978)
de Boer et al., Proc. Natl. Acad. Sci. USA, 80:21 (1983)
De Louvencourt et al., J. Bacteriol., 154:737 (1983)
Dijkema et al., EMBO J., 4:761 (1985)
Dower et al., Nuc. Acids Res., 16: 6127 (1988)
Dykes et al., Eur. J. Biochem., 174: 411 (1988)
EPO Publ. Nos. 036 259 and 063 953
EPO Publ. Nos. 036 776, 136 829 and 136 907
EPO Publ. No. 121 775
EPO Publ. No. 127 328
EPO Publ. Nos. 127 839 and 155 476
EPO Publ. No. 164 556
EPO Publ. No. 267 851
EPO Publ. No. 329 203
Felgner et al., Proc. Natl. Acad. Sci., 84:7413 (1987)
Feigner et al., J. Biol. Chem., 269:2550 (1994)
Fiedler et al., Anal. Biochem, 170: 38 (1988)
Forsberg et al., Biofactors, 2: 105-112, (1989)
Forsberg et al., Int. J. Protein Chem., 11: 201-211, (1992)
Franke and Hruby, J. Gen. Virol., 66:2761 (1985)
Fraser et al., In Vitro Cell. Dev. Biol., 25:225 (1989)
Freifelder, Physical Biochemistry: Applications to Biochemistry and Molecular Biology, W.H. Freeman and Co., 2nd edition, New York, N.Y. (1982).
Friesen et al., “The Regulation of Baculovirus Gene Expression”, in: The Molecular Biology of Baculoviruses (ed. Walter Doerfler), 1986
Gaillardin et al., Curr. Genet., 10:49 (1985)
Ghrayeb et al., EMBO J., 3: 2437 (1984)
Gleeson et al., J. Gen. Microbiol., 132:3459 (1986)
Gluzman, Cell, 23:175 (1981)
Goeddel et al., Nuc. Acids Res., 8:4057 (1980)
Gorman et al., Proc. Natl. Acad. Sci. USA, 79:6777 (1982b)
Graham and van der Eb, Virology, 52:456 (1973)
Gram et al., Bio/Technology, 12: 1017-1023, (1994)
Guan et al., Gene, 67:21 (1997)
Harlander, “Transformation of Streptococcus lactis by electroporation”, in: Streptococcal Genetics (ed. J. Ferretti and R Curtiss III), (1987)
Henikoff et al., Nature, 283:835 (1981); Hollenberg et al., Curr. Topics Microbiol. Immunol., 96:119 (1981)
Hinnen et al., Proc. Natl. Acad. Sci. USA, 75:1929 (1978)
Hollenberg et al., “The Expression of Bacterial Antibiotic Resistance Genes in the Yeast Saccharomyces cerevisiae”, in: Plasmids of Medical, Environmental and Commercial Importance (eds. K. N. Timmis and A. Puhler), 1979
Ito et al., J. Bacteriol., 153:163 (1983)
Kaufman et al., Mol. Cell. Biol., 9:946 (1989)
Kawai and Nishizawa, Mol. Cell. Biol., 4:1172 (1984)
Knott et al., Eur. J. Biochem., 174: 405-410, (1988)
Kunkel, Proc. Natl. Acad. Sci. USA, 82:488, (1985)
Kunkel et al., Methods in Enzymol., 154:367 (1987)
Kunze et al., J. Basic Microbiol., 25:141 (1985)
Kurtz et al., Mol. Cell. Biol., 6:142 (1986)
Kushner, “An improved method for transformation of Escherichia coli with ColE1-derived plasmids”, in: Genetic Engineering: Proceedings of the International Symposium on Genetic Engineering (eds. H. W. Boyer and S. Nicosia), (1978)
Labouesses B., Bull. Soc. Chim. Biol., 42: 1293, (1960)
Lebacq-Verheyden et al., Mol. Cell. Biol., 8: 3129 (1988)
Luckow and Summers, Virology, 17:31 (1989)
Maeda et al., Nature, 315:592 (1985)
Mandel et al., J. Mol. Biol., 53: 159 (1970);
Maniatis et al., Sciences, 236:1237 (1987)
Marcus, Int. J. Peptide Protein Res., 25: 542-546, (1985)
Martin et al., DNA, 7: 99 (1988)
Marunoto et al., J. Gen. Virol., 68:2599 (1987)
Masson et al., FEMS Microbiol. Lett., 60: 273 (1989)
Masui et al., in: Experimental Manipulation of Gene Expression, (1983)
McCarroll and King, Curr. Opin. Biotechnol., 8:590 (1997)
Mercerau-Puigalon et al., Gene, 11:163 (1980)
Miller et al., Ann. Rev. Microbiol., 42:177 (1988)
Miller et al., Proc. Natl. Acad. Sci. USA, 8: 856 (1988)
Miller et al., Bioessays, 4:91 (1989)
Mitchell W., Meth. of Enzymol., 47: 165-170 (1977)
Mitchell, W. M, Harrington, W. F., J. of Biol. Chem., 243 (18): 4683-4692 (1968)
Miyajima et al., Gene, 58: 273 (1987)
Moks et al., Bio/Technology, 5: 379-382, (1987)
Myanohara et al., Proc. Natl. Acad. Sci. USA, 80:1 (1983)
Neuman et al, EMBO J., 1:841 (1982)
Oka et al., Proc. Natl. Acad. Sci. USA, 82: 7212 (1985)
Orr-Weaver et al., Methods in Enzymol., 101:228 (1983)
Palva et al., Proc. Natl. Acad. Sci. USA, 79: 5582 (1982)
Panthier et al., Curr. Genet., 2:109 (1980)
Perry et al., Infec. Immun., 32: 1295 (1981)
PCT Publ. No. WO 84/04541
PCT Pub. No. WO 89/046699
Piers et al., Gene, 134: 7, (1993)
Pilon et al., Biotechnol. Prog., 13, 374-379 (1997)
Powell et al., Appl. Environ. Microbiol., 54: 655, (1988)
Raibaud et al., Ann. Rev. Genet., 18:173 (1984)
Ray et al., Bio/Technology, 11: 64 (1993)
Rine et al., Proc. Natl. Acad, Sci. USA, 80:6750 (1983)
Roggenkamp et al., Mol. Gen. Genet., 202:302 (1986)
Sambrook and Russell, Molecular Cloning: A Laboratory Manual, 3rd edition (Jan. 15, 2001) Cold Spring Harbor Laboratory Press, ISBN: 0879695765
Sanford et al., Methods Enzymol., 217:483 (1993)
Sassone-Corsi and Borelli, Trends Genet., 2:215 (1986)
Schellenberger et al., Int. J. Peptide Protein Res., 41: 326(1993)
Shen, Proc. Nat'l. Acad. Sci., (USA), 281: 4627 (1984)
Shimatake et al., Nature, 292:128 (1981)
Shinmizu et al., Mol. Cell. Biol., 6:1074 (1986)
Shine et al., Nature, 254: 34, (1975).
Smith et al., Proc. Natl. Acad. Sci. USA, 82: 8404 (1985.)
Smith et al., Mol. Cell. Biol., 3:2156 (1983)
Somkuti et al., Proc. 4th Eur. Cong. Biotechnology, 1: 412 (1987)
Steitz et al., “Genetic signals and nucleotide sequences in messenger RNA”, in: Biological Regulation and Development: Gene Expression (ed. R. F. Goldberger) (1979) Studier et al., J. Mol. Biol., 189:113 (1986)
Stinchcomb et al., J. Mol. Biol., 158:157 (1982)
Summers and Smith, Texas Agricultural Experiment Station Bulletin No. 1555, 1987.
Tabor et al., Proc. Natl. Acad. Sci. USA, 82:1074 (1985)
Taketo, Biochim. Biophys. Acta, 949: 318 (1988)
U.S. Pat. No. 4,336,336
U.S. Pat. No. 4,551,433
U.S. Pat. No. 4,689,406
U.S. Pat. No. 4,738,921
U.S. Pat. No. 4,745,056
U.S. Pat. Nos. 4,837,148 and 4,929,555
U.S. Pat. No. 4,873,192
U.S. Pat. Nos. 4,876,197 and 4,880,734
U.S. Pat. No. 5,595,887 to Coolidge et at.
U.S. Pat. No. 5,707,826 to Wagner et al.
U.S. Pat. No. 6,316,224
Vaheri and Pagano, Virology, 27:434 (1965)
Van den Berg et al., Bio/Technology, 8:135 (1990)
VanDevanter et al., Nucleic Acids Res., 12:6159 (1984)
Vlak et al., J. Gen. Virol., 69:765 (1988)
Walker and Gaastra, eds., Techniques in Molecular Biology, MacMillan Publishing Company, New York (1983)
Wang et al., J. Bacteriol., 172: 949 (1990)
Watson, Molecular Biology of the Gene, 4th edition, Benjamin/Cummings Publishing Company, Inc., Menlo Park, Calif. (1987)
Weissmann, “The cloning of interferon and other mistakes”, in: Interferon 3 (ed. I. Gresser), 1981
Williams et al., Control of Drosophila wine and haltere development by the nuclear vestigial gene product, Genes Dev. Dec. 5, (12B):2481-95 (1991)
Wright, Nature, 321: 718 (1986)
Yelverton et al., Nuc. Acids Res., 9:731 (1981)
Zimmerman, Biochem. Biophys. Acta., 694:227 (1982)

All publications, patents and patent applications cited herein and priority U.S. patent application 60/383,214 are incorporated herein by reference. The foregoing specification has been described in relation to certain embodiments thereof, and many details have been set forth for purposes of illustration, however, it will be apparent to those skilled in the art that the invention is susceptible to additional embodiments and that certain of the details described herein may be varied considerably without departing from the basic principles of the invention.

1. A method for cleaving a peptide bond of a polypeptide, comprising:
combining the polypeptide with clostripain;
wherein the polypeptide contains a GLP-1 (7-36) amino acid sequence having at its C-terminus at least a fragment having an amino acid sequence of Formula I
-Xaa₁-Xaa₂-Xaa₃- (I);
Xaa₁ is a residue of aspartic acid, glycine, proline or glutamic acid;
Xaa₂ is an arginine residue at position 36;
Xaa₃ is any amino acid residue other than an acidic amino acid residue.

2. A method for producing a desired peptide from a polypeptide, comprising:
combining the polypeptide and clostripain, wherein the polypeptide comprises Formula (II)
(Xaa₃-Peptide₁-Xaa₁-Xaa₂)ₙ-Xaa₃-Peptide₁-Xaa₁-Xaa₂ (II);
the desired peptide is Xaa₃-Peptide₁-Xaa₁-Xaa₂ having the sequence of GLP-1 (7-36)
n is an integer ranging from 0 to 50;
Xaa₁ is aspartic acid, glycine, proline or glutamic acid;
Xaa₂ is arginine;
Xaa₃ is not an acidic amino acid.

3. A method for producing a GLP-1(7-36) peptide, comprising the steps of
(a) obtaining a polypeptide of the Formula VI:
Tag-Linker-[GLP-1 (7-36)]_{q} Formula VI
wherein,
Tag is a translation initiation sequence having SEQ ID NO:17 or 18;
Linker is a cleavable peptide linker of Formula IV described above; GLP-1(7-36) has SEQ ID NO:1; and
q is an integer of about 2 to about 20;
(b) combining the polypeptide of Formula VI and clostripain.

4. A method for producing a GLP-1(7-36)NH₂ peptide having SEQ ID NO:2, comprising the steps of
(a) obtaining a polypeptide of the Formula VIII:
Tag-Linker-[GLP-1(7-36)-Linker₂]_{q} VIII
wherein:
Tag is a translation initiation sequence comprising SEQ ID NO:17 or 18;
Linker is a cleavable peptide linker having Formula IV:
(Peptide₅)ₘ-Xaa₁-Xaa₂ IV
wherein:
n is an integer ranging from 0 to 50;
m is an integer ranging from 0 to 50;
Xaa₁ is aspartic acid, glycine, proline or glutamic acid;
Xaa₂ is arginine; and
Peptide₅ is a single or pair of amino acid residues;
Linker₂ is SEQ ID NO:23;
GLP-1(7-36) has SEQ ID NO:1;
q is an integer of about 2 to about 20;
(b) combining the polypeptide of Formula VIII and clostripain in the presence of ammonia.

5. A method for producing a GLP-1(7-37) peptide having SEQ ID NO:3, comprising the steps of
(a) obtaining a polypeptide of the Formula VIII:
Tag-Linker-[GLP-1(7-36)-Linker₂]_{q} VIII
wherein:
Tag is a translation initiation sequence comprising SEQ ID NO:17 or 18;
Linker is a cleavable peptide linker having Formula IV:
(Peptide₅)ₘ-Xaa₁-Xaa₂ IV
wherein:
n is an integer ranging from 0 to 50;
m is an integer ranging from 0 to 50;
Xaa₁ is aspartic acid, glycine, proline or glutamic acid;
Xaa₂ is arginine; and
Peptide₅ is a single or pair of amino acid residues;
Linker₂ is SEQ ID NO:23;
GLP-1(7-36) has SEQ ID NO:1;
q is an integer of about 2 to about 20;
(b) combining the polypeptide of Formula VIII and clostripain in the presence of glycine.

6. A method for producing a GLP-1(7-36)(K26R)—NH₂ peptide having SEQ ID NO:6, comprising:
(a) obtaining a polypeptide of the Formula VIII:
Tag-Linker-[GLP-1(7-36)(K26R)-Linker₂]_{q} VIII
wherein:
Tag is a translation initiation sequence comprising SEQ ID NO:17 or 18;
Linker is a cleavable peptide linker having Formula IV:
(Peptide₅)ₘ-Xaa₁-Xaa₂ IV
wherein:
n is an integer ranging from 0 to 50;
m is an integer ranging from 0 to 50;
Xaa₁ is aspartic acid, glycine, proline or glutamic acid;
Xaa₂ is arginine; and
Xaa₄ and Xaa₅ are separately any amino acid;
GLP-1(7-36)(K26R) has SEQ ID NO:5;
q is an integer of about 2 to about 20;
(b) combining the polypeptide of Formula VIII and clostripain in the presence of ammonia.

7. The method of claim 1, 2, 3, 4, 5 or 6 wherein the polypeptide is a soluble polypeptide.

8. The method of claim 1, 2, 3, 4, 5 or 6 wherein the combining step performed at about 15° C. to about 25° C.

9. The method of claim 1, 2, 3, 4, 5 or 6 wherein the combining step is performed between a pH of about 5 to about 11.

10. The method of claim 1, 2, 3, 4, 5 or 6 wherein the concentration of clostripain is about 0.01 to about 3.0 units of clostripain per about 2 to about 5 mg polypeptide.

11. The method of claim 1, 2, 3, 4, 5 or 6 wherein the combining step is performed in the presence of about 0.5 mM to about 10 mM CaCl₂.

12. The method of claim 3, 4, 5 or 6 wherein the Linker comprises Pro-Gly-Xaa₁-Xaa₂, and wherein Xaa₁ is aspartic acid and Xaa₂ is arginine.

13. The method of claim 3, 4, 5 or 6 wherein the Linker comprises Val-Asp-Xaa₁-Xaa₂, and wherein Xaa₁ is aspartic acid and Xaa₂ is arginine.

14. The method of claim 3, 4, 5 or 6 wherein the Linker comprises Ile-Thr-Xaa₁-Xaa₂(SEQ ID NO:26), Gly-Ser-Xaa₁-Xaa₂ (SEQ ID NO:25), Cys-His-Xaa₁-Xaa₂ (SEQ ID NO:14), Cys-His Xaa-Xaa-Xaa₁-Xaa₂ (SEQ ID NO:15), Gly-Ser-Glu-Xaa₂ (SEQ ID NO:16), Val-Asp-Xaa₁-Xaa₂ (SEQ ID NO:24) and wherein Xaa₁ is aspartic acid and Xaa₂ is arginine.

15. A desired peptide produced by the method of any one of claims 1, 2, 3, 4, 5 or 6.

16. The method of claim 1, 2, 3, 4, 5 or 6 wherein the peptide is continuously removed by performing the cleavage reaction in a chamber having a filtration membrane, wherein the membrane allows the peptide to pass through but does not permit the polypeptide or the clostripain to pass through.

17. A method of producing a peptide from a polypeptide comprising:
a) obtaining bacterial inclusion bodies containing the polypeptide;
b) solubilizing the polypeptide within the bacterial inclusion bodies using urea;
c) combining the polypeptide and clostripain in the optional presence of up to about 8 M urea using clostripain,
wherein the polypeptide contains a site of Formula I:
Xaa₁-Xaa₂-Xaa₃ (I)
Xaa₁ is aspartic acid, glycine, proline or glutamic acid;
Xaa₂ is arginine; and
Xaa₃ is not an acidic amino acid.

18. A method for producing a GLP-1 (7-36) peptide from a polypeptide comprising:
a) obtaining bacterial inclusion bodies containing the polypeptide comprising Formula II
(Xaa₃-Peptide₁-Xaa₁-Xaa₂)ₙ-Xaa₃-Peptide₁-Xaa₁-Xaa₂ (II)
wherein
the GLP-1 (7-36) peptide has the Formula Xaa₃-Peptide₁-Xaa₁-Xaa₂;
n is an integer ranging from 0 to 50;
Xaa₁ is aspartic acid, glycine, proline or glutamic acid;
Xaa₂ is arginine; and
Xaa₃ is an histidine;
b) solubilizing the polypeptide within the bacterial inclusion bodies using urea;
c) combining the polypeptide and clostripain in the optional presence of up to about 8 M urea.

19. A method for producing a GLP-1 (7-36) peptide from a polypeptide using clostripain, which comprises:
a) obtaining bacterial inclusion bodies containing the polypeptide comprising Formula III
(Linker-Xaa₃-Peptide₁)ₙ-Linker-Xaa₃-Peptide₁ (III)
wherein:
n is an integer ranging from 0 to 50;
the GLP-1 (7-36) peptide comprises Xaa₃-Peptide₁
Xaa₃ is histidine;
Linker is a cleavable peptide linker having Formula IV:
(Peptide₅)ₘ-Xaa₁-Xaa₂ IV
n is an integer ranging from 0 to 50;
m is an integer ranging from 0 to 50;
Xaa₁ is aspartic acid, glycine, proline or glutamic acid;
Xaa₂ is arginine; and
Peptide₅ is any disposable amino acid sequence;
b) solubilizing the polypeptide within the bacterial inclusion bodies using urea; and,
c) combining the polypeptide and clostripain in the optional presence of up to about 8 M urea.

20. The method of claim 17, 18 or 19 wherein the combining step is performed at about 40° C. to about 50° C.

21. The method of claim 17, 18 or 19 wherein the combining step is performed between a pH of about 8.5 to about 9.7.

22. The method of claim 17, 18 or 19 wherein the concentration of clostripain is about 10 to about 30 units clostripain per about 1 mg polypeptide.

23. The method of claim 17, 18 or 19 wherein the concentration of polypeptide is about 1.5 to about 15 mg/mL.

24. The method of claim 17, 18 or 19 wherein the combining step is performed in the presence of 0.5 mM to about 10 mM CaCl₂.

25. The method of claim 17, 18 or 19 wherein the combining step is performed in the presence of about 0.5 to about 3.0 mM cysteine.

26. The method of claim 17, 18 or 19 wherein the combining step is performed in the presence of glycine thereby generating a peptide that has a C-terminal glycine during the cleavage reaction.

27. The method of claim 17, 18 or 19 wherein the combining step is performed in the presence of Gly-Leu, thereby generating a peptide with Gly-Leu at the C-terminal end during the cleavage reaction.

28. The method of claim 17, 18 or 19 wherein the combining step is performed in the presence of ammonia to generate a peptide with a C-terminal amide.

29. The method of claim 28 wherein the ammonia is present at about 1 M to about 5 M.

30. The method of claim 17, 18 or 19 wherein the peptide is continuously removed from the cleavage reaction.

31. The method of claim 30 wherein the peptide is continuously removed by performing the cleavage reaction in a chamber having a filtration membrane, wherein the membrane allows the peptide to pass through but does not permit the polypeptide or the clostripain to pass through.

32. The method of claim 19 wherein the Linker comprises Pro-Gly-Xaa₁-Xaa₂ (SEQ ID NO:27), and wherein Xaa₁ is aspartic acid and Xaa₂ is arginine.

33. The method of claim 19 wherein the Linker comprises Val-Asp-Xaa₁-Xaa₂ (SEQ ID NO:24), and wherein Xaa₁ is aspartic acid and Xaa₂ is arginine.

34. The method of claim 19 wherein the Linker comprises Ile-Thr-Xaa₁-Xaa₂ (SEQ ID NO:26), and wherein Xaa₁ is aspartic acid and Xaa₂ is arginine.

35. The method of claim 13 wherein the cleavage is performed in the presence of about 0.5 to about 3.0 mM cysteine.

36. The method of claim 3, 4, 5, 6, 17, 18 or 19 wherein the obtaining step include recombinant production of the polypeptide.


SEQUENCE LISTING (31 sequences)

SEQ ID NO: 1 - protein, 30 aa, Artificial Sequence
  A synthetic peptide
HAEGTFTSDVSSYLEGQAAKEFIAWLVKGR

SEQ ID NO: 2 - protein, 30 aa, Artificial Sequence
  A synthetic peptide
  SITE 30: Xaa = Arg-NH2
HAEGTFTSDVSSYLEGQAAKEFIAWLVKGX

SEQ ID NO: 3 - protein, 31 aa, Artificial Sequence
  A synthetic peptide
HAEGTFTSDVSSYLEGQAAKEFIAWLVKGRG

SEQ ID NO: 4 - protein, 31 aa, Artificial Sequence
  A synthetic peptide
  SITE 31: Xaa = Gly- NH2
HAEGTFTSDVSSYLEGQAAKEFIAWLVKGRX

SEQ ID NO: 5 - protein, 30 aa, Artificial Sequence
  A synthetic peptide
HAEGTFTSDVSSYLEGQAAREFIAWLVKGR

SEQ ID NO: 6 - protein, 30 aa, Artificial Sequence
  A synthetic peptide
  SITE 30: Xaa = Arg-NH2
HAEGTFTSDVSSYLEGQAAREFIAWLVKGX

SEQ ID NO: 7 - protein, 31 aa, Artificial Sequence
  A synthetic peptide
HAEGTFTSDVSSYLEGQAAREFIAWLVKGRG

SEQ ID NO: 8 - protein, 31 aa, Artificial Sequence
  A synthetic peptide
  SITE 31: Xaa = Gly-NH2
HAEGTFTSDVSSYLEGQAAREFIAWLVKGRX

SEQ ID NO: 9 - protein, 117 aa, Artificial Sequence
  A synthetic peptide
  SITE 1: Xaa = T7-tag
XGSGPGDRHAEGTFTSDVSSYLEGQAAKEFIAWLVKGRAFLGPGDRHAEGTFTSDVSSYLEGQAAKEFIAWLVKGRAFLGPGDRHAEGTFTSDVSSYLEGQAAKEFIAWLVKGRAFL

SEQ ID NO: 10 - protein, 34 aa, Artificial Sequence
  A synthetic peptide
  SITE 34: Xaa = HSe (homoserine)
HAEGTFTSDVSSYLEGQAAKEFIAWLVKGRAFAX

SEQ ID NO: 11 - protein, 277 aa, Artificial Sequence
  A synthetic peptide
  SITE 1: Xaa = T7 tag
XMHAEGTFTSDVSSYLEGQAAKEFIAWLVKGRAFAMHAEGTFTSDVSSYLEGQAAKEFIAWLVKGRAFAMHAEGTFTSDVSSYLEGQAAKEFIAWLVKGRAFAMHAEGTFTSDVSSYLEGQAAKEFIAWLVKGRAFAMHAEGTFTSDVSSYLEGQAAKEFIAWLVKGRAFAMHAEGTFTSDVSSYLEGQAAKEFIAWLVKGRAFAMHAEGTFTSDVSSYLEGQAAKEFIAWLVKGRAFAMHAEGTFTSDVSSYLEGQAAKEFIAWLVKGRAFAMHAE

SEQ ID NO: 12 - protein, 34 aa, Artificial Sequence; identical to SEQ ID NO: 10
  A synthetic peptide
  SITE 34: Xaa = Hse-lactone

SEQ ID NO: 13 - protein, 119 aa, Artificial Sequence
  A synthetic peptide
  SITE 1: Xaa = T7-tag
XGSGPGDRHAEGTFTSDVSSYLEGQAAREFIAWLVKGRKRAFLGPGDRHAEGTFTSDVSSYLEGQAAREFIAWLVKGRAFLGPGDRHAEGTFTSDVSSYLEGQAAREFIAWLVKGRAFL

SEQ ID NO: 14 - protein, 4 aa, Artificial Sequence
  A synthetic peptide
CHDR

SEQ ID NO: 15 - protein, 6 aa, Artificial Sequence
  A synthetic peptide
  SITE 3, 4: Xaa = any amino acid
CHXXDR

SEQ ID NO: 16 - protein, 4 aa, Artificial Sequence
  A synthetic peptide
GSER

SEQ ID NO: 17 - protein, 11 aa, Artificial Sequence
  A synthetic T7 tag
ASMTGGQQMGR

SEQ ID NO: 18 - protein, 12 aa, Artificial Sequence
  A synthetic T7 tag
MASMTGGQQMGR

SEQ ID NO: 19 - protein, 29 aa, Artificial Sequence
  A synthetic inclusion body leader partner
GSGQGQAQYLSASCVVFTNYSGDTASQVD

SEQ ID NO: 20 - protein, 38 aa, Artificial Sequence
  A synthetic inclusion body leader partner that is a part of the Drosophila      vestigial polypeptide (Vg)
GSGQGQAQYLAASLVVFTNYSGDTASQVDVNGPRAMVD

SEQ ID NO: 21 - protein, 35 aa, Artificial Sequence
  A synthetic inclusion body leader partner that is a part of polyhedrin polypeptide (Ph)
GSAEEEEILLEVSLVFKVKEFAPDAPLFTGPAYVD

SEQ ID NO: 22 - protein, 22 aa, Artificial Sequence
  A synthetic inclusion body leader partner that includes a part of the lactamase polypeptide
SIQHFRVALIPFFAAFSLPVFA

SEQ ID NO: 23 - protein, 8 aa, Artificial Sequence
  A synthetic cleavable peptide linker sequence
AFLGPGDR

SEQ ID NO: 24 - protein, 4 aa, Artificial Sequence
  A synthetic cleavable peptide linker sequence
VDDR

SEQ ID NO: 25 - protein, 4 aa, Artificial Sequence
  A synthetic cleavable peptide linker sequence
GSDR

SEQ ID NO: 26 - protein, 4 aa, Artificial Sequence
  A synthetic cleavable peptide linker sequence
ITDR

SEQ ID NO: 27 - protein, 4 aa, Artificial Sequence
  A synthetic cleavable peptide linker sequence
PGDR

SEQ ID NO: 28 - protein, 70 aa, Artificial Sequence
  A synthetic peptide
HGRHAEGTFTSDVSSYLEGQAAKEFIAWLVKGRGRHGRHAEGTFTSDVSSYLEGQAAKEFIAWLVKGRGR

SEQ ID NO: 29 - protein, 8 aa, Artificial Sequence
  A synthetic peptide
  SITE 1: Xaa = GLP-1 (GLP-1 = Glucagon like peptides)
XAFLGPDR

SEQ ID NO: 30 - skipped

SEQ ID NO: 31 - protein, 122 aa, Artificial Sequence
  A synthetic peptide
  SITE 14: Xaa = Peptide 5
ASMTGGQQMGRGSXDRHAEGTFTSDVSSYLEGQAAKEFIAWLVKGRAFLGPGDRHAEGTFTSDVSSYLEGQAAKEFIAWLVKGRAFLGPGDRHAEGTFTSDVSSYLEGQAAKEFIAWLVKGR